(19) United States
(12) Patent Application Publication
(10) Pub. No.: US 2008/0227689 A1
(43) Pub. Date: Sep. 18, 2008

(54) Dampening Humoral and Innate Immunity by Inhibition of Ppgalnact-1

(76) Inventors: Jamey Marth, San Diego, CA (US); Mari Tenno, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/088,226

(22) PCT Filed: Sep. 29, 2006
(86) PCT No.: PCT/US06/38437

§ 371 Date: Mar. 26, 2008

Related U.S. Application Data
(60) Provisional application No. 60/722,314, filed on Sep. 29, 2005.

(51) Int. Cl. A61K 38/00; A61K 31/70; C12Q 1/48; A61P 37/00

(52) U.S. Cl. 514/2; 514/44; 435/15

(57) ABSTRACT

The invention provides methods and compositions for treating pathogenic immune and inflammatory disorders through inhibition of polypeptide GalNAc transferase 1 (ppGalNAcT-1) transferase activity.

### CROSS-REFERENCES TO RELATED APPLICATIONS

This application claims the benefit of U.S. Provisional Patent Application No. 60/722,314, filed on Sep. 29, 2005, the entire contents of which is hereby incorporated herein by reference.

### STATEMENT AS TO RIGHTS TO INVENTIONS MADE UNDER FEDERALLY SPONSORED RESEARCH AND DEVELOPMENT

This invention was made with Government support under Grant No. DK48247, awarded by the National Institutes of Health. The Government has certain rights in this invention.

### FIELD OF THE INVENTION

The present invention relates to the field of reducing or inhibiting undesirable immune responses, particularly through inhibition of polypeptide GalNAc transferase 1 (ppGalNAcT-1) activity.

### BACKGROUND OF THE INVENTION

Protein O-glycosylation in the secretory pathway of cells produces a highly abundant posttranslational modification comprised of a diverse repertoire of extracellular O-glycan structures. Sometimes referred to as mucin-type O-glycans, their origination depends upon the enzymatic transfer of N-acetylgalactosamine (GalNAc) to serine and threonine residues in the context of polypeptide sequences (McGuire, E. J. et al., J. Biol. Chem. 242:3745-3755 (1967); Elhammer, A. et al., J. Biol. Chem. 261:5249-5255 (1986); Wang Y. et al., J. Biol. Chem. 267:12709-12716 (1992); Van den Steen, P. et al., Crit. rev. Biochem. Mol. Biol. 33:151-208 (1998)). This initial step is followed in the Golgi apparatus by the transfer of additional saccharide linkages determined by the expression and substrate specificity properties of other glycosyltransferases operating more distally in the O-glycan biosynthetic pathway (Schachter, H. et al., Symp. Soc. Exp. Biol. 43:1-26 (1989); Brockhausen, I. “In Carbohydrates in Chemistry and Biology”, Volume 3 (New York: Wiley-VCH), pp. 313-328 (2000)). Genomic information on polypeptide GalNAc transferase (ppGalNAcT) structure and expression followed biochemical purification of ppGalNAcT activity and the acquisition of amino acid sequences (Hagen, F. K. et al., J. Biol. Chem. 268:18960-18965 (1993); Homa, F. L. et al., J. Biol. Chem. 268:12609-12616 (1993)). Multiple cDNA sequences encoding ppGalNAcT isozymes were subsequently discovered, while genetic disruption of a homologous ppGalNAcT locus in the mouse failed to ablate O-glycan formation (Hennet, T. et al., Proc. Natl. Acad. Sci. USA 92:12070-12074 (1995); Sorensen, T. et al., J. Biol. Chem. 270:24166-24173 (1995); White, T. et al., J. Biol. Chem. 270:24156-24165 (1995); Marth, J. D. Glycobiology 6:701-705 (1996); Zhang, Y. et al., J. Biol. Chem. 278:573-584 (2003)). An unexpectedly large family of ppGalNAcTs is now evident among multi-cellular organisms thus far analyzed with mammals expressing at least 15 ppGalNAcT isozymes (Ten Hagen, K. G. et al., Glycobiology 13:1 R-16R (2003); Cheng, L. et al., FEBS Lett. 566:17-24 (2004)).

Comparative analyses of ppGalNAcT isozymes indicate that not all are created equal and each may have unique physiologic functions. Besides considerable differences in tissue- and cell-type expression patterns, substrate specificities among polypeptide of ppGalNAcT isozymes are also varied (Nehrke, K. et al., Glycobiology 8:367-371 (1998); Young, W. W. et al., Glycobiology 13:549-557 (2003); Pratt, M. R. et al., Chem. Biol. 11:1009-1016 (2004)). Preferences for dissimilar polypeptide sequences have been observed along with the existence of a hierarchical process in protein O-glycosylation that reflects the dependence of some ppGalNAcTs on adjacent O-linked GalNAc linkages produced previously by other isozymes (Bennet, E. P. et al., J. Biol. Chem. 273:30472-30481 (1998); Ten Hagen, et al., J Biol Chem. 274:27867 (1999). Hanisch, F-G. et al., J. Biol. Chem. 274:9946-9954 (1999); Hanisch, F-G. et al., Glycobiology. 11:731-40 (2001); Kato, K. et al., Biochem. Biophys. Res. Comm. 287:110-115 (2001)). These findings indicate that limitations likely exist in the potential for functional redundancy among ppGalNAcT isozymes, as would also be predicted by the level of orthologous ppGalNAcT gene sequence conservation in vertebrate radiation. And yet mammalian models of ppGalNAcT deficiency thus far studied, including ppGalNacT-4, -5, and -13, lack physiologic alterations linked with decreased O-glycan formation indicative of vital endogenous roles (Hennet, T. et al., Proc. Natl. Acad. Sci. USA 92:12070-12074 (1995); Ten Hagen, K. G. et al., Glycobiology 13:1 R-16R (2003); Zhang, Y. et al., J. Biol. Chem. 278:573-584 (2003)). Further investigations are warranted however and mutations in the human GALNT3 gene encoding ppGalNAcT-3 have been described in familial tumoral calcinosis which may result in reduced protein O-glycosylation (Topaz, O. et al., Nat. Genet. 36:579-581 (2004); Ichikawa, S. et al., J. Clin. Endocrinol. Metab. 90:2420-2423 (2005)).

The only mammalian O-glycan structures identified with endogenous physiologic activity at present comprise distinct Core 1 and Core 2 type O-glycans that contribute to Selectin ligand dependent control of leukocyte trafficking and in the maintenance of peripheral CD8+ T cell homeostasis by inhibition of apoptosis (Priatel, J. J. et al., Immunity 12:273-283 (2000); Lowe, J. B., et al., Annu. Rev. Biochem. 72:643-691 (2003); Rosen, S. D. Annu. Rev. Immunol. 22:129-156 (2004)). Nevertheless, other endogenous functions may exist and are perhaps indicated by developmental abnormalities among invertebrate model organisms bearing alterations in O-glycan formation (Schwientek, T. et al. J. Biol. Chem. 277:22623-22638 (2002); Ten Hagen, K. G. et al., J. Biol. Chem. 277:22616-22622 (2002)). The potential to discover critical biological activities that are dependent upon specific ppGalNAcT isozymes remains high in genetic research on model organisms, and may be the only approach to eventually comprehend the need for multiple ppGalNAcT isozymes throughout the evolution of multi-cellular organisms.

Polypeptide GalNAcT-1 is the prototypical ppGalNAcT family member and is highly expressed among most tissues and cell types (Homa, F. L. et al., J. Biol. Chem. 268:12609-12616 (1993); Marth, J. D. Glycobiology 6:701-705 (1996); Kingsley, P. D. et al., Glycobiology 10:1317-1323 (2000); Young, W. W. et al., Glycobiology 13:549-557 (2003)). This isozyme exhibits a selective preference for polypeptide sequences (Brockhausen, I. et al., Glycoconj. J. 13:849-856 (1996); Wandall, H. H. et al., J. Biol. Chem. 272:23503-23514 (1997); Gerken, T. A. et al., Biochem. 43:9888-9900 (2004)) further suggesting a key function is likely provided by its expression among cells of intact organisms. In order to detect physiologic activities linked to ppGalNAcT-1, we have generated and characterized mice lacking this distinct isozyme. We find that although ppGalNAcT-1 deficiency is generally tolerated and does not cause lethality or infertility, a defect in the formation of selectin ligands invariably occurs resulting in lymphocytosis associated with a major deficit in B lymphocyte localization to lymph nodes and attenuation of immunoglobulin-G production. Depressed E- and P-selectin ligand formation on neutrophils is also observed with markedly decreased recruitment during inflammation. These features of ppGalNAcT-1 function reveal a unique profile of selectin ligand formation exposing an essential physiologic role in directing cell type-specific leukocyte trafficking and sustaining optimal immune responses.

### BRIEF SUMMARY OF THE INVENTION

The present invention provides compounds and methods for ameliorating a pathogenic immune or inflammatory response in a mammal in need thereof by decreasing or inhibiting ppGalNAcT-1 transferase activity in the mammal. This decrease in activity can be attained in a number of ways including, but not limited to, administering a compound that directly or indirectly decreases or inhibits the enzymatic activity of the ppGalNAcT-1. The compound can be, for example, an inactivating substrate analog of a ppGalNAcT-1, for example, an analog of a donor saccharide (i.e, a UDP-N-acetylgalactosamine), and/or an analog of an acceptor polypeptide. ppGalNAcT-1 activity can also be decreased or inhibited through a decrease in transcription or translation of a ppGalNAcT-1 gene, a decrease in the RNA stability and/or half-life of a ppGalNAcT-1 transcript, and a decrease in stability and/or half-life of a ppGalNAcT-1 translated product. Agents that decrease or inhibit expression and/or the function of a ppGalNAcT-1 enzyme are thus of use in the methods of the invention. In certain embodiments, the methods preferentially decrease or inhibit the enzymatic activity of a ppGalNAcT-1 enzyme in comparison to the inhibition of other ppGalNAc isozymes.

The invention further provides screening methods for identifying agents for use in reducing immune and/or inflammatory disorders through in vitro and in vivo assays using a ppGalNAcT-1 gene, or functional segments thereof. This includes in vitro assays using a nucleic acid sequence encoding a ppGalNAcT-1 enzyme or amino acid sequences having ppGalNAcT-1 enzymatic activity, as well as in vivo assays using animal model systems, including mammalian model systems.

Accordingly, in a first aspect, the invention provides methods of inhibiting a pathogenic inflammatory or immune response in a mammal comprising administering to the mammal a compound that inhibits polypeptide GalNAc transferase 1 (ppGalNAcT-1) activity.

In some embodiments, the compound is a competitive inhibitor of a ppGalNAcT-1 substrate. In some embodiments, the compound comprises a ppGalNAcT-1 substrate analog. In some embodiments, the substrate analog is an analog of a donor substrate, for example, an analog of UDP-GalNAc, UDP or GalNAc. In some embodiments, the substrate analog is an analog of a peptide acceptor substrate. In some embodiments, the analog of an acceptor substrate is a peptidomimetic. In some embodiments, the compound is a non-competitive inhibitor.

In some embodiments, the compound is an inhibitory nucleic acid, for example, a small interfering RNA molecule (siRNA), a micro RNA molecule (miRNA), an antisense RNA molecule, or a ribozyme.

In some embodiments, the mammal is a human. In some embodiments, the mammal is a domestic mammal, for example, a canine, a feline, a rodent.

In some embodiments, the pathogenic immune response is an autoimmune disorder, for example, rheumatoid arthritis, multiple sclerosis, myasthenia gravis, autoimmune uveitis, or systemic lupus erythematosus.

In another aspect, the invention provides methods of identifying a compound for inhibiting a pathogenic immune or inflammatory disorder in a mammal. In one embodiment, the screening methods comprise:
a) providing an assay mixture which comprises: a polypeptide GalNAc transferase 1 (ppGalNAcT-1), a potential immune or inflammatory response inhibitor, a UDP-GalNAc donor saccharide, an acceptor polypeptide, and additional reagents required for ppGalNAcT-1 transferase activity;
b) incubating the assay mixture under conditions in which the ppGalNAcT-1 is active;
c) determining whether the amount of N-acetylgalactosamine transferred to the acceptor polypeptide is decreased in comparison to an assay mixture which lacks the potential immune or inflammatory response inhibitor; and
d) determining whether the potential immune or inflammatory response inhibitor decreases a pathogenic immune or inflammatory disorder in a mammalian disease model for the disorder, whereby a compound for use in inhibiting a pathogenic immune or inflammatory disorder in a mammal is identified.

In another embodiment, the screening methods comprise:
a) providing a cell which comprises a polynucleotide that encodes a ppGalNAcT-1, an acceptor polypeptide for the ppGalNAcT-1, and UDP-GalNAc;
b) contacting the cell with a potential immune or inflammatory response inhibitor and incubating the cell under conditions in which the ppGalNAcT-1 is normally expressed;
c) determining whether the level of a target O-linked glycan moiety is decreased compared to the target O-linked glycan level in the absence of the potential immune or inflammatory response inhibitor; and
d) determining whether the potential immune or inflammatory response inhibitor decreases a pathogenic immune or inflammatory disorder in a mammalian disease model for the disorder, whereby a compound for use in inhibiting a pathogenic immune or inflammatory disorder in a mammal is identified.

In one embodiment, the target O-linked glycan moiety is sialyl 6-sulfo Lewis x or sialyl Lewis x.

### DEFINITIONS

The terms “UDP-N-acetyl-D-galactosamine:polypeptide N-acetylgalactosaminyl-transferase 1” or “polypeptide GalNAc transferase 1” or “polypeptide N-acetylgalactosaminyltransferase 1” or “ppGalNAcT-1” interchangeably refer to the isozyme type 1 of the enzyme that catalyzes the initial reaction in O-linked oligosaccharide biosynthesis, classified under EC 2.4.1.41. Specifically, ppGalNAcT-1 catalyzes the transfer of an N-acetyl galactosamine residue from the nucleotide sugar UDP-N-acetylgalactosamine donor substrate to a serine or threonine residue on a polypeptide acceptor substrate. A ppGalNAcT-1 shares at least 90% amino acid sequence identity, for example, 95%, 96%, 97%, 98% or 99% sequence identity, with the amino acid sequence for human ppGalNAcT-1 available through GenBank under accession number NP_{—}065207 or AAC50327. A ppGalNAcT-1 shares at least 90% nucleic acid sequence identity, for example, 95%, 96%, 97%, 98% or 99% sequence identity, with the nucleic acid sequence for human ppGalNAcT-1 available through GenBank under accession number NM_{—}020474, Y10343, U41514 or X85018. In some embodiments, a ppGalNAcT-1 preferentially transfers an N-acetyl galactosamine residue to a serine or threonine residue on a polypeptide acceptor substrate containing an amino acid sequence sharing at least 90% amino acid sequence identity, for example, 95%, 96%, 97%, 98% or 99% sequence identity, to one or more of the following the amino acid sequences: Ac-(S/T)PPP, PPDAA(S/T)AAPL, PPDAA(S/T)AAPLR, PHMAQV(S/T)VGPGL, GVVP(S/T)VVPG, PRFQDSSSKAPPPLPSPSRLPG, AHGVTSAPDTR, APPAHGVTSAPDTRPAPGC, RPAPGSTAPPA, PDTRPAPGSTAPPAC, TAPPAHGVTSAPDTRPAPGSTAPP, PTTTPISTTTMVTPTPTPTC, Ac-SAPTTSTTSAPT, and LSESTTQLPGGGPGCA.

The term “ppGalNacT-1 activity” refers to the transfer of an N-acetyl galactosamine residue (or analog thereof) from a nucleotide sugar UDP-N-acetylgalactosamine donor substrate (or analog thereof) to a serine or threonine residue on a polypeptide acceptor substrate (or analog thereof). The extent of activity can be measured by any means known in the art, for example, using an anion exchange solid phase in reverse phase high performance liquid chromatography (HPLC).

The terms “decrease” or “reduce” or “inhibit” interchangeably refer to the detectable reduction of a measured response (e.g., enzymatic activity, immune response, inflammatory response, immune cell trafficking). The decrease, reduction or inhibition can be partial, for example, at least 10%, 25%, 50%, 75%, or can be complete (i.e., 100%). The decrease, reduction or inhibition can be measured in comparison to a control. For example, decreased, reduced or inhibited responses can be compared before and after treatment. Decreased, reduced or inhibited responses can also be compared to an untreated control, or to a known value.

The term “substrate analog” refers to a compound (e.g., small molecules) that shares structural and/or functional similarity with an enzyme substrate, but unlike the enzyme substrate, the substrate analog inhibits the function of the enzyme upon binding. A substrate analog can have structural similarity to an enzyme substrate as measured on a 2-dimensional or 3-dimensional (electron densities, location of charged, uncharged and/or hydrophobic moieties) basis. A substrate analog can have functional similarity with an enzyme substrate inasmuch as the substrate analog binds to the enzyme. A substrate analog can be a competitive inhibitor.

The term “donor substrate” refers to a substrate that provides a moiety to be transferred by a transferase enzyme to an acceptor substrate. A donor substrate of ppGalNAcT-1 can be UDP-N-acetylgalactosamine or an analog thereof.

The term “acceptor substrate” refers to a substrate to which a transferase covalently attaches a moiety transferred from a donor substrate. An acceptor substrate of ppGalNAcT-1 is a polypeptide sequence or a peptidomimetic thereof.

The phrase “sequence identity,” in the context of two nucleic acids or polypeptides, refers to two or more sequences or subsequences that have a certain level of nucleotide or amino acid residue identity, when compared and aligned for maximum correspondence, as measured using one of the following sequence comparison algorithms or by visual inspection. Preferably, the aligned sequences share at least 90% sequence identity, for example, at least 95%, 96%, 97%, 98%, 99% or 100% sequence identity. The sequence identity can exist over a region of the sequences that is at least about 10, 20 or 50 residues in length, sometimes over a region of at least about 100 or 150 residues. In some embodiments, the sequences share a certain level of sequence identity over the entire length of the sequence of interest.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (on the World Wide Web at ncbi.nhn.nih.gov/) (see Henikoff & Henikoff, Proc. Natl. Acad. Sci. USA 89:10915 (1989)).

A “compound that inhibits ppGalNAcT-1 activity” refers to any compound that inhibits ppGalNAcT-1 activity. The inhibition can be, for example, on the transcriptional, translational or enzymatic level. Accordingly, the compound can be in any chemical form, including nucleic acid or nucleotide, amino acid or polypeptide, monosaccharide or oligosaccharide, nucleotide sugar, or small organic molecule.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” as used herein applies to amino acid sequences. One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

### BRIEF DESCRIPTION OF THE DRAWINGS

FIG. 1 shows that ppGalNAcT-1 Mutagenesis and Loss of Enzyme Activity by Deletion of Exon 3. (A) Construction of the ppGalNAcT-1 targeting vector for subsequent Cre-loxP recombination. (B) Cre recombination results in the deletion of exon 3 producing the null allele (type 1) or the flanking of exon 3 by loxP sites to generate the floxed allele (type 2). Restriction enzyme sites for A and B are indicated (A-Apa I, B-Bam HI, R-Eco RV, X-Xho I, Sp-Spe I, N-Not 1). PCR primer binding sites (P1, P2, and P3) are indicated by arrows. (C) Genomic Southern blot analysis of targeted ES Cell clones (6-6, 1-4, and 3-3) using the loxP probe. (D) Genomic Southern blotting of tail DNA using the genomic probe indicates the presence of germline Type 1 and Type 2 mutations in the gene encoding ppGalNAcT-1. (E) Enzyme activity was assayed on total protein extracts from various tissues using the peptide substrate PRFQDSSSKAPPPLPSPSRLPG. O-glycosylated products were profiled by anion exchange chromatography and evaluated by reverse phase HPLC. Data are represented as the mean ±SDV from three separate experiments. SG, salivary gland; Spl, spleen; Thy, thymus. (F) Polypeptide GalNAcT-1 cDNA structures as sequenced from the kidney tissue of mice bearing either wild-type or deleted (A) alleles. (G) ppGalNAcT enzyme activity towards the EA2 peptide (PTTDSTTPAPTTK) in COS7 cell extracts following transfection of cDNAs expressed by the pIMKF3 vector. Data are represented as the mean ±SDV from three separate experiments.

FIG. 2 shows elevated blood lymphocyte levels with reduced lymph node cell number and diminished protein O-glycosylation. (A) Leukocyte counts were obtained from blood of wild-type and ppGalNAcT-1 deficient mice. Cell numbers are expressed per μl of whole blood ±SEM (n=21 of each genotype) (WBC, white blood cells; Neu, neutrophils; Lym, lymphocytes). An unpaired t test indicated significance of ***p<0.001, **p<0.01. (B) Leukocyte abundance was also determined among various hematopoietic organs including inguinal lymph nodes (ILN), axillary lymph nodes (ALN), cervical lymph nodes (CLN), mesenteric lymph nodes (MLN), Peyer's patch (PP), spleen (Spl), thymus (Thy), and bone marrow (BM). Data are means ±SEM from twelve mice of each genotype. An unpaired t test indicated significance of ***p<0.001, **p<0.01. (C) B lymphocyte numbers (CD19+) measured among lymphoid aggregates. (D) T lymphocyte numbers (CD3+) among lymphoid aggregates. Data are means ±SEM from twelve mice of each genotype.

FIG. 3 shows selective deficiency in lymph node L-selectin ligand expression. (A) Frozen sections of inguinal and mesenteric lymph nodes incubated with CD4-FITC, CD8-FITC, and B220-Rho, were visualized by fluorescent microscopy (100× magnification). (B) Unaltered expression level of L-selectin on CD19+ or CD3+ lymphocytes in spleen, blood, inguinal, and mesenteric lymph nodes determined by flow cytometry. Dotted lines represent background cell fluorescence using isotype-specific control antibodies. (C) Frozen sections of inguinal and mesenteric lymph nodes stained with L-selectin-IgG chimera and MECA-79 antibody (400× magnification). (D) Relative levels of L-selectin ligands and MECA-79 expression on HEV were obtained by quantifying fluorescent signals from serial and parallel tissue sections of inguinal lymph nodes (ILN) and mesenteric lymph nodes (MLN) using deconvolution microscopy and MetaMorph software analysis (see Experimental Procedures).

FIG. 4 shows ppGalNAcT-1 directs lymphocyte homing to specific lymph nodes. CMFDA-labeled lymphocytes (2.5×107) obtained from wild-type mice were injected into the tail vein of recipients of indicated genotypes. Lymphoid aggregates as denoted above were harvested (A) 1 hour or (B) 24 hours after injection. CMFDA+ T and B lymphocytes were quantified by flow cytometry. Data are means ±SEM from eight mice of each genotype. An unpaired t test indicated significance of ***p<0.001, **p<0.01, *p<0.05.

FIG. 5 shows normal antigen receptor activation contrasts with attenuated antibody production due to loss of ppGalNAcT-1. (A) Serum Ig levels among 8-week-old naive mice (n=16). Points represent measurements from individual animals. The median Ig levels are depicted as horizontal bars (mean ±SEM). An unpaired t test indicated significance of ***p<0.001, *p<0.05. (B) The expression levels of activation markers (CD44, B7.2, and I-Ab) and CD40 were analyzed by flow cytometry on CD 19+ lymphocytes derived from spleen (Spl), peripheral lymph nodes (PLN), and mesenteric lymph nodes (MLN). The results shown are representative of three separate experiments. Dotted lines represent fluorescence of cells stained using an isotype control antibody. (C) B lymphocytes were isolated and stimulated by antibody to IgM or LPS. The proliferation response is measured by 3H-thymidine incorporation. Data are represented as the mean ±SEM from three mice of the indicated genotype. (D) Anti-DNP antibody levels produced in response to immunization with 10 μg of T-independent antigen DNP-Ficoll measured at indicated times.

FIG. 6 shows that reduced neutrophil O-Glycosylation and E- and P-selectin ligands in ppGalNAcT-1 deficiency attenuate Gr-1+ cell recruitment and inflammation. (A) Reduced protein O-glycosylation detected by PNA lectin binding and flow cytometry on Gr-1+ cells in circulation. (B) Reduced E- and P-selectin ligands among circulating Gr-1+ cells detected by selectin-IgM chimera binding and flow cytometry. Loss of selectin-IgM chimera binding in the presence of EDTA is also shown. Data are representative of three separate experiments. (C) The expression levels of adhesion molecules (CD11a, CD11b, CD18, and CD62L) or selectin counter-receptors (CD24 and PSGL-1) on Gr-1+ cells in blood were analyzed by flow cytometry. The results shown are representative of three separate experiments. Dotted lines show fluorescent signals of cells stained using an isotype control antibody. (D) Peritoneal cells were collected and Gr-1+ neutrophils were counted prior to (0 hr) or after (2 or 4 hr) intraperitoneal injection of casein to initiate acute peritonitis. Data are represented as the mean ±SEM from eight mice of the indicated genotype (***p<0.001).

FIG. 7 shows a comparison of mouse and human ppGalNAcT-1 sequences. (A) Genomic sequence spanning exon 3 of the mouse gene encoding ppGalNAcT-1. Upper reference genomic sequence was obtained from The National Center for Biotechnology Information (BLAST; Galnt1; accession number NT_{—}039674). The lower sequence was obtained from mouse 129/SvJ strain-derived genomic clone 14-1 which represents the targeted allele. Exon-intron boundaries are denoted by arrows and were previously described (Bennett et al., J. Biol. Chem. (1998) 273: 30472-30481). Exon sequences are shown in uppercase; flanking intron sequences are shown in lowercase. (B) Comparison of mouse and human ppGalNAcT-1 and human ppGalNAcT-13 in exon 3 amino acid sequence. Differences in amino acid sequences are denoted in bold with asterisks below.

### DETAILED DESCRIPTION

1. Introduction

It has been discovered that ppGalNAcT-1 glycosyltransferase is a key determinant in the synthesis of L-selectin ligands among peripheral lymph nodes and the majority of E- and P-selectin ligands produced by neutrophils. ppGalNAcT-1 supports lymph node residency among the majority of B lymphocytes and its deficiency markedly impairs immunoglobulin-G production in pre- and post-immunization. ppGalNAcT-1 further enables the majority of neutrophil trafficking in early inflammation. The initiation of O-glycan formation by ppGalNAcT-1 is a key determinant in sustaining humoral and innate immunity in part by increasing selectin ligand expression levels that support B lymphocyte retention among peripheral lymph nodes and neutrophil recruitment during inflammation. Accordingly, the present invention provides methods and agents for treating undesirable immune and inflammatory responses.

The present invention provides methods and compounds that are suitable for ameliorating (e.g., decreasing, inhibiting or preventing) rogue immune responses and rogue inflammatory responses. Such methods and compounds can be used, prophylactically, chronically or acutely, to reduce, inhibit or prevent undesirable immune or inflammatory responses. Also provided are screening methods for identifying compounds that are useful for decreasing, inhibiting or preventing undesirable immune or inflammatory responses. Such compounds are suitable for use directly, or for use as lead compounds to identify further compounds that are useful for decreasing, inhibiting or preventing rogue immune or inflammatory responses. Transgenic animals that lack a functional gene for a ppGalNAcT-1 are also provided by the invention. In a preferred embodiment of the invention, the methods preferentially decrease, inhibit or prevent the transferase activity of ppGalNAcT-1 in comparison to other ppGalNAcT isozymes.

The invention is based in part on the surprising discovery that mice with a non-functional ppGalNAcT-1 gene exhibit reduced immune and inflammatory responses. Accordingly, the methods of the invention include reducing, inhibiting or preventing undesirable immune and/or inflammatory responses by administering to a mammal in need thereof a therapeutically effective amount of one or more compounds that decrease or inhibit ppGalNAcT-1 activity. The compounds can reduce or prevent the synthesis of, or enhance the degradation of, selectin ligands on substrate glycoproteins. The compounds can reduce or inhibit the transcription or translation of a ppGalNAcT-1 gene, thereby reducing or inhibiting the production of a functional ppGalNAcT-1 enzyme. The compounds can directly or indirectly decrease or inhibit the enzymatic activity of a ppGalNAcT-1 enzyme. In some embodiments, the methods of the invention cause a change in the activity of a ppGalNAcT-1 enzyme. In certain embodiments, the compounds are substrate analogs of a ppGalNAcT-1 enzyme, for example, analogs of a donor sugar, nucleotide or nucleotide-sugar, or analogs of an acceptor polypeptide. The mammal can be a non-human mammal, including canine, feline, porcine, bovine, ovine, murine, rodentia and lagomorpha. The mammal is typically a human.

The invention also includes blocking agents that decrease or inhibit the activity of a ppGalNAc-T, particularly a ppGalNAcT-1 enzyme. The blocking agents of the invention can act directly on the enzyme, or act as a substrate for the enzyme, for instance as an inactivating substrate analog of the enzyme. The blocking agents can also decrease or inhibit the expression of a gene that encodes the enzyme, at either or both the transcriptional and translational levels.

Methods are also disclosed for preparing the immune and/or inflammatory response modulating agents as well as various screening assays to identify suitable candidates. Therapeutic and other uses for these compounds are also provided.

2. Methods for Decreasing ppGalNAcT-1-Mediated Immune/Inflammatory Responses

The present invention provides methods for inhibiting pathogenic immune and/or inflammatory responses. Certain undesirable immune and/or inflammatory response are mediated in part by O-glycan selectin ligands that require functional ppGalNAcT-1 for their synthesis (e.g., sialyl 6-sulfo Lewis x and sialyl Lewis x). By decreasing, inhibiting or preventing the synthesis of O-glycan selecting ligands on cells, undesirable immune and/or inflammatory responses can be reduced, inhibited or prevented.

A. Inhibitors of ppGalNAcT-1

In one embodiment, the methods involve reducing, inhibiting or preventing an undesirable immune and/or inflammatory response by inhibiting the enzymatic activity of an a ppGalNAcT enzyme, particularly a ppGalNAcT-1 isozyme. Preferably, the inhibitory agent preferentially inhibits the enzymatic activity of a ppGalNAcT-1 enzyme in comparison to inhibiting the enzymatic activity of other ppGalNAcT isozymes, for example, ppGalNAcT-2, -3, -4, -5, -6, -7, -8, -9, -10, -11, -12 or -13.

Having identified the target enzyme to be inhibited (i.e., a ppGalNAcT-1 enzyme), many approaches can be used to block its activity. Examples of agents capable of inhibiting enzyme activity include substrate analogs, suicide substrates, alkylating agents, and inhibitory nucleic acids (reviewed in Ferscht, Structure and Mechanism in Protein Science: A Guide to Enzyme Catalysis and Protein Folding, 3rd Edition, 1999, W.H. Freeman & Co.). The methods of decreasing, inhibiting or preventing ppGalNAcT-1 activity can involve administering to a subject, including a mammal such as a human, a compound that is an analog of a substrate for a ppGalNAcT-1, including a donor nucleotide-sugar, nucleotide or sugar, and/or an acceptor polypeptide or peptidomimetic. In certain embodiments, the methods involve decreasing, inhibiting or preventing ppGalNAcT-1 activity by administering to a subject an analog of a preferential substrate for a ppGalNAcT-1 in comparison to other ppGalNAcT-1 isozymes.

Naturally occurring molecules which show inhibitory effects can also be isolated for use in the present invention. The biosynthesis of glycoproteins or glycolipids is a complex metabolic pathway that depends on many factors for regulation. Naturally occurring inhibitory compounds can be purified and used to further inhibit activity.

The preferred glycosyltransferase inhibitors of the present invention have the ability to cross the cell membrane and enter the Golgi apparatus. Thus, the blocking agents are preferably sufficiently hydrophobic to allow diffusion through the membrane. Generally, they have no other adverse effects on cellular metabolism, so that other glycosylation reactions proceed while the specific reaction is inhibited. The blocking agents are preferably relatively small molecules, thereby avoiding immunogenicity and allowing passage through the cell membrane. Ideally, they have a molecular weight of between about 100-2000 daltons, but may have molecular weights up to 5000 or more, depending upon the desired application. In most preferred embodiments, the inhibitors have molecular weights of between about 200-600 daltons.

The inhibitors of the present invention preferably have strong affinity for the target enzyme, so that at least about 60-70% inhibition of ppGalNAcT-1 activity is achieved, more preferably about 75%-85% and most preferably 90%-95% or more. In some embodiments, the inhibitors will completely inhibit ppGalNAcT-1 activity. The affinity of the enzyme for the inhibitor is preferably sufficiently strong that the dissociation constant, or Kᵢ, of the enzyme-inhibitor complex is less than about 10⁻⁵ M, typically between about 10⁻⁶ and 10⁻⁸ M.

Enzyme inhibition generally involves the interaction of a substance with an enzyme so as to decrease the rate of the reaction catalyzed by that enzyme. Inhibitors can be classified according a number of criteria. For example, they may be reversible or irreversible. An irreversible inhibitor dissociates very slowly, if at all, from its target enzyme because it becomes very tightly bound to the enzyme, either covalently or noncovalently. Reversible inhibition, in contrast, involves an enzyme-inhibitor complex which may dissociate.

Inhibitors can also be classified according to whether they are competitive, noncompetitive or uncompetitive inhibitors. In competitive inhibition for kinetically simple systems involving a single substrate, the enzyme can bind either the substrate or the inhibitor, but not both. Typically, competitive inhibitors resemble the substrate or the product(s) and bind the active site of the enzyme, thus blocking the substrate from binding the active site. A competitive inhibitor diminishes the rate of catalysis by effectively reducing the affinity of the substrate for the enzyme. Typically, an enzyme may be competitively inhibited by its own product because of equilibrium considerations. Since the enzyme is a catalyst, it is in principle capable of accelerating a reaction in the forward or reverse direction.

Noncompetitive inhibitors allow the enzyme to bind the substrate at the same time it binds the inhibitor. A noncompetitive inhibitor acts by decreasing the turnover number of an enzyme rather than diminishing the proportion of free enzyme. Another possible category of inhibition is mixed or uncompetitive inhibition, in which the inhibitor affects the binding site and also alters the turnover number of the enzyme. Enzyme inhibition of kinetically complex systems involving more than one substrate, as can be the case for ppGalNAcT enzymes, are described in Segel, Enzyme Kinetics, (Wiley, N.Y. 1975).

ppGalNAcT-1 activity and its inhibition or enhancement is typically assayed according to standard methods for determining enzyme activity. For a general discussion of enzyme assays, see, Rossomando, “Measurement of Enzyme Activity” in Guide to Protein Purification, Vol. 182, Methods in Enzymology (Deutscher ed., 1990). Assays for the measurement of ppGalNAcT-1 transferase activity are described herein, and for example, in Wandall, et al., J Biol Chem (1997) 272:23503-14 and Tenno, et al., J Biol Chem (2002) 277:47088-96. An assay for ppGalNAcT-1 transferase activity typically contains a buffered solution adjusted to physiological pH, a source of divalent cations (e.g., Mn²⁺ and/or Ca²⁺), a donor substrate (for example, a labeled UDP-N-acetylgalactosamine moiety), an acceptor substrate (e.g., an appropriate peptide or polypeptide sequence, as discussed herein), ppGalNAcT-1 enzyme, and the sample or fraction of a sample whose inhibitory activity is to be tested. After a predetermined time at 23° C. or 37° C., for example, 0.5, 1.0, 1.5 or 2.0 hours, the reaction is stopped and the glycosylated product is isolated and measured according to standard methods (e.g., in a scintillation counter, by ion exchange chromatography). When testing for the ability of a test compound to decrease or inhibit ppGalNAcT-1 transferase activity, the transferase activity of a ppGalNAcT-1 exposed to the test compound is compared to the transferase activity of a ppGalNAcT-1 in a control unexposed to the test compound.

1. Donor Substrate Analogs

The donor substrate of ppGalNAcT are sugar nucleotides, usually diphosphonucleosides. For example, uridine diphosphosugars are donor substrates for the formation of O-glycosylated proteins by ppGalNAcT enzymes. In some embodiments, the inhibitor is an analog of a donor substrate, e.g., an analog of uridine diphosphate (UDP), N-acetylgalactosamine, or UDP-N-acetylgalactosamine.

Using this knowledge, one of skill in the art can readily synthesize a number of sugar nucleotides which can then be tested to identify those capable of maximum inhibition of a specific enzyme. The term “sugar nucleotide” or “nucleotide sugar” as used herein refers both to sugar nucleotides discussed above and to various analogs thereof that might be synthesized or isolated from natural sources. The number of variations on this structure is limitless. For instance, both the ester linkage between the sugar and phosphate and the anhydride linkage of the pyrophosphate are potential targets of enzymatic cleavage. Replacement of the O—P or C—O linkage with a more stable C—P bond provides nucleotide monophosphate or diphosphate sugar analogs that are more resistant to enzymatic degradation. Such compounds have the potential to selectively inhibit glycoprotein or glycolipid synthesis by acting as substrate analogs of a ppGalNACT-1 enzyme. See, e.g., Vaghefi et al., J. Med. Chem. 30:1383-1391 (1987), and Vaghefi et al., J. Med. Chem. 30:1391-1399 (1987).

Another approach is to replace the monophosphate or diphosphate bridge between the sugar residue and the nucleoside moiety. For instance, the diphosphate bridge can be replaced with an isosteric —OCONHSO₂O— residue. See, Samarasa, et al., J. Med. Chem. 28:40-46 (1985).

Analogs of sugar nucleotides capable of inhibiting glycosylation have been used as antibiotics and antiviral agents. Examples of such compounds include 2-deoxy-D-glucose, which is transformed to either UDP-2dGlc or GDP-2dGlc and in that form inhibits glycosylation of glycoproteins in the viral envelope. DeClercq, Biochem. J. 205:1 (1982), which is hereby incorporated herein by reference. Antibiotics such as tunicamycin and streptovirudin are also effective because of their ability to inhibit glycosylation. For instance, tunicamycin is an analog of UDP-GlcNAc, the donor substrate for N-acetylglucosaminyltransferases. The replacement of diphosphate bridge with a carbon chain allows tunicamycin to cross the cell membrane but still readily bind the active site of the enzyme. Examples of uridine analog drugs are disclosed in Chapter 52 of Hardman and Limbird, Goodman and Gilman's The Pharmacological Basis of Therapeutics, 10th Edition, 2001, McGraw-Hill, the disclosure of which is hereby incorporated herein by reference. The structure of these and related compounds provide one of skill in the art with direction in designing and synthesizing compounds with similar inhibitory effects in accordance with the present invention as described herein.

Nucleotides are the byproduct of the reaction by which N-acetylgalactosamine residues are transferred to acceptor substrates. Nucleotides have been found to competitively inhibit ppGalNAcT enzymes. Thus, various nucleotides and their analogs have potential as inhibitors of these enzymes. For example, analogs of uridine monophosphate (UMP), uridine diphosphate (UDP) or uridine triphosphate (UTP) can be used to inhibit ppGalNAcT-1 activity. Sugar analogs can also serve as donor substrate inhibitors. Galactosamine, acetylgalactosamine, N-acetylgalactosamine, and analogs thereof, can be used to inhibit the enzymatic activity of ppGalNAcT-1. For example, a benzyl-substituted N-acetyl galactosamine, for example, α-D-GalNAc-1-benzyl, can be used to inhibit the activity of ppGalNAcT-1 (see, for example, Hassan, et al., J Biol Chem (2000) 275:38197-205).

In some embodiments, sugar nucleotide analogs are administered to inhibit the enzymatic activity of ppGalNAcT-1. Sugar nucleotide inhibitors of ppGalNAcT enzymes, including ppGalNAcT-1, are known in the art. For example, uridine 5′-phosphoric (1-hexadecanesulfonic) anhydride inhibits a GalNAc transferase and O-glycosylation (Hatanaka, et al., Biochem Biophys Res Commun (1991) 175:668-72). UDP-GalNAc analogs with C-glycosidic hydroxymethylene linkages between the sugar and nucleoside moieties can also be used to inhibit ppGalNAcT activity (Schafer and Thiem, J Org Chem (2000) 65:24-29). In another strategy, replacing the 2-acetamido group of a GalNAc moiety with an N-acyl, N-alkyl, N-haloalkyl, N-haloacyl group, or an azido group can inhibit ppGalNAcT activity (Lazarevic and Thiem, Carbohydr Res (2002) 337:2187-94; and Lazarevic and Thiem, Carbohydr Res (2006) 341:569-76). In a different approach, O-methylated UDP-GalNAc analogs can be synthesized as ppGalNAc inhibitors (Busca, et al., (2003) Bioorg Med Chem Lett 13:1853-6).

2. Acceptor Substrate Analogs

In addition to the donor substrate analogs, analogs of acceptor substrates (e.g., modified peptides and peptidomimetics) can also be used as inhibitors. Again, the skilled artisan will recognize a variety of possible structures that can be used. Because of the acceptor peptide substrate specificity ppGalNAcT-1, specific inhibition of a ppGalNAcT-1 transferase reaction can be achieved. Ideally, the inhibitory compounds should be capable of acting as specific acceptor substrates for ppGalNAcT-1, even in the presence of other enzymes, including other ppGalNAcT isozymes. In addition, the compound should be an efficient acceptor substrate. Suitable acceptor substrates for inhibition of a ppGalNAcT-1 transferase reaction include polypeptides comprising an amino acid sequence recognized by ppGalNAcT-1, polypeptides comprising a modified amino acid sequence recognized by ppGalNAcT-1, and peptidomimetics.

In some embodiments, the polypeptides are peptides that are from 4 to 25 amino acids in length, for example 4, 5, 6, 7, 8, 9, 10, 11, 12, 13, 14, 15, 16, 17, 18, 19, 20, 21, 22, 23, 24 or 25 amino acids in length. The peptides can comprise a naturally recognized amino acid sequence or an amino acid sequence with one or more modifications (e.g., additions, deletions, substitutions). For example, one or more amino acid residues can be substituted with a same or different D-amino acid residue, or a non-naturally occurring amino acid residue. In particular, one or more of the serine or threonine target residues can be deleted or modified, for example, with a corresponding D-amino acid residue, or with a different D- or L-amino acid residue, for example, a glycine, an alanine, an asparagine, or an aspartic acid, or with a non-naturally occurring amino acid (see, for example, Wragg, et al., J Biol Chem (1995) 270:16947-954).

Exemplified polypeptide sequences comprising acceptor substrates for ppGalNAcT-1 include, for example, apomucin and apo A1 (see, for example, Conde, et al., Neurochem Res (2004) 22:483-490; and Tenno, et al., J Biol Chem (2002) 277:47088-96). Exemplified peptide sequences of use as inhibitory acceptor substrates of ppGalNAcT-1 include, for example, Ac-XPPP, PPDAAXAAPL, PPDAAXAAPLR, PHMAQVXVGPGL, GVVPXVVPG, and PRFQDSSSKAPPPLPSPSRLPG (wherein at least one S is X), wherein X is a deleted amino acid residue or any D- or L-amino acid, naturally occurring or non-naturally occurring, other than a L-serine or L-threonine residue. In some embodiments, X is a D- or L-glycine, alanine, asparagine or aspartic acid. See, for example, Wragg, et al., supra; Tenno, et al., J Biol Chem (2002) 277:47088-96. Additional peptide sequences of use as inhibitory acceptor substrates of ppGalNAcT-1 include, for example, AHGVTSAPDTR, APPAHGVTSAPDTRPAPGC, RPAPGSTAPPA, PDTRPAPGSTAPPAC, TAPPAHGVTSAPDTRPAPGSTAPP, PTTTPISTTTMVTPTPTPTC, Ac-SAPTTSTTSAPT, and LSESTTQLPGGGPGCA, wherein one or more of S or T residues are deleted or replaced with another D- or L-amino acid, naturally occurring or non-naturally occurring, other than a L-serine or L-threonine residue. In some embodiments, S or T are replaced with a D- or L-glycine, alanine, asparagine or aspartic acid. See, for example, Wandall, et al., J Biol Chem (1997) 272:23503-14. Inhibitory acceptor substrates decrease or inhibit the glycosylation of a native acceptor substrate by ppGalNAcT-1. Inhibitory acceptor substrate polypeptide or peptide sequences can contain conservatively substituted amino acid residues in comparison to the paradigm amino acid sequence, provided above. In some embodiments, the inhibitory acceptor substrate polypeptide or peptide sequences share at least 90% sequence identity, for example, 95%, 96%, 97%, 98%, 99% sequence identity, with the exemplified amino acid sequences.

In some embodiments, the inhibitory acceptor substrate is a peptidomimetic. As used herein, the terms “peptidomimetic” and “mimetic” refer to a synthetic chemical compound that has substantially the same structural characteristics of an acceptor polypeptide or peptide of ppGalNAcT-1, but that functions to inhibit the transferase activity of the enzyme. Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987), which are incorporated herein by reference). Peptide mimetics that are structurally similar to therapeutically useful peptides can be used to produce an equivalent or enhanced inhibitory effect on ppGalNAcT-1 transferase activity. Generally, peptidomimetics are structurally similar to a paradigm polypeptide (i.e., a polypeptide or peptide sequence that serves as an acceptor substrate), such as the (poly)peptides exemplified in this application, but have one or more peptide linkages optionally replaced by a linkage selected from the group consisting of, e.g., —CH₂NH—, —CH₂S—, —CH₂—CH₂—, —CH═CH—(cis and trans), —COCH₂—, —CH(OH)CH₂—, and —CH₂SO—. The mimetic can be either entirely composed of synthetic, non-natural analogues of amino acids, or can be a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of binding to a ppGalNAcT-1 enzyme and inhibiting the glycosylation of a native acceptor substrate. Further guidance for designing peptidomimetics is described, for example, in U.S. Pat. No. 7,105,488, hereby incorporated herein by reference.

B. Inhibition of ppGalNAcT-1 Gene Expression

Decreasing or inhibiting ppGalNAcT-1 gene expression can be achieved through the use of inhibitory nucleic acids (e.g., small interfering RNA (siRNA), micro RNA (miRNA), antisense RNA, ribozymes, etc.). Inhibitory nucleic acids can be single-stranded nucleic acids that can specifically bind to a complementary nucleic acid sequence. By binding to the appropriate target sequence, an RNA-RNA, a DNA-DNA, or an RNA-DNA duplex or triplex is formed. Such inhibitory nucleic acids can be in either the “sense” or “antisense” orientation. See, for example, Tafech, et al., Curr Med Chem (2006) 13:863-81; Mahato, et al., Expert Opin Drug Deliv (2005) 2:3-28; Scanlon, Curr Pharm Biotechnol (2004) 5:415-20; and Scherer and Rossi, Nat Biotechnol (2003) 21:1457-65.

In one embodiment, the inhibitory nucleic acid can specifically bind to a target nucleic acid sequence or subsequence that encodes a ppGalNAcT-1. Administration of such inhibitory nucleic acids can decrease or inhibit undesirable immune or inflammatory responses by reducing or eliminating the transfer of a N-acetylgalactosamine to a polypeptide acceptor. Nucleotide sequences encoding a ppGalNAcT-1 are known for several species, including human. Human nucleic acid sequences encoding ppGalNAcT-1 have been published as GenBank accession numbers NM_{—}020474, Y10343, U41514 and X85018. Nucleotide sequences from non-human species encoding a ppGalNAcT-1 have been published as GenBank accession numbers XM_{—}001135802 and XM_{—}523910 (Pan troglodyte), XM_{—}001105040 (Macaca mulatta), XM_{—}537284 and XM_{—}861664 (Canis familiaris), D85389 (Sus sp.-porcine), NM_{—}177519 (Bos taurus), and BC090962, NT_{—}039674 and NM_{—}013814 (Mus musculus). From these nucleotide sequences, one can derive a suitable inhibitory nucleic acid.

1. Antisense Oligonucleotides

In some embodiments, the inhibitory nucleic acid is an antisense molecule. Antisense oligonucleotides are relatively short nucleic acids that are complementary (or antisense) to the coding strand (sense strand) of the mRNA encoding a ppGalNAcT-1. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a ppGalNAcT-1. Accordingly, antisense oligonucleotides decrease the expression and/or activity of ppGalNAcT-1.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO 88/09810) or the blood-brain barrier (see, e.g., PCT Publication No. WO 89/10134), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide can be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an -anomeric oligonucleotide. An anomeric oligonucleotide forms specific double-stranded hybrids with complementary RNA in which, contrary to the usual-units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The selection of an appropriate oligonucleotide can be readily performed by one of skill in the art. Given the nucleic acid sequence encoding a ppGalNAcT-1, one of skill in the art can design antisense oligonucleotides that bind to a target nucleic acid sequence and test these oligonucleotides in an in vitro or in vivo system to confirm that they bind to and mediate the degradation of the mRNA encoding the ppGalNAcT-1. To design an antisense oligonucleotide that specifically binds to and mediates the degradation of a ppGalNAcT-1 encoding nucleic acid, it is preferred that the sequence recognized by the oligonucleotide is unique or substantially unique to the ppGalNAcT-1 to be inhibited. For example, sequences that are frequently repeated across an encoding sequence may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a ppGalNAcT-1.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

Antisense inhibition of ppGalNAcT-1 has been described, for example, in Adachi, et al., J Immunol (1997) 159:2645-51.

2. Small Interfering RNA (siRNA or RNAi)

In some embodiments, the inhibitory nucleic acid is a small interfering RNA (siRNA or RNAi) molecule. RNAi constructs comprise double stranded RNA that can specifically block expression of a target gene. “RNA interference” or “RNAi” is a term initially applied to a phenomenon where double-stranded RNA (dsRNA) blocks gene expression in a specific and post-transcriptional manner. RNAi provides a useful method of inhibiting gene expression in vitro or in vivo. RNAi constructs can include small interfering RNAs (siRNAs), hairpin RNAs, and other RNA species which can be cleaved in vivo to form siRNAs. RNAi constructs herein also include expression vectors (“RNAi expression vectors”) capable of giving rise to transcripts which form dsRNAs or hairpin RNAs in cells, and/or transcripts which can produce siRNAs in vivo.

RNAi expression vectors express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell.

The RNAi constructs contain a nucleotide sequence that hybridizes under physiologic conditions of the cell to the nucleotide sequence of at least a portion of the mRNA transcript for the gene to be inhibited (i.e., a ppGalNAcT-1 encoding nucleic acid sequence). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity can be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, for example, 95%, 96%, 97%, 98%, 99%, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodie-sters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Exemplary RNAi constructs that specifically recognize a particular gene, or a particular family of genes can be selected using methodology outlined in detail above with respect to the selection of antisense oligonucleotide. Similarly, methods of delivery RNAi constructs include the methods for delivery antisense oligonucleotides outlined in detail above.

3. Ribozymes

In some embodiments, the inhibitory nucleic acid is a ribozyme. Ribozymes molecules designed to catalytically cleave an mRNA transcripts can also be used to prevent translation of mRNA (See, e.g., PCT International Publication WO 90/11364; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; WO 88/04300; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

3. Screening Methods

One can identify lead compounds that are suitable for further testing to identify those that are therapeutically effective inhibitory agents by screening a variety of compounds and mixtures of compounds for their ability to decrease or inhibit ppGalNAcT-1 activity and prevent or inhibit rogue immune responses or inflammatory responses. The testing can be performed using a suitable polypeptide or peptide acceptor substrate, to which an N-acetylgalactosamine residue can be added.

The use of screening assays to discover naturally occurring compounds with desired activities is well known and has been widely used for many years. For instance, many compounds with antibiotic activity were originally identified using this approach. Examples of such compounds include monolactams and aminoglycoside antibiotics. Compounds which inhibit various enzyme activities have also been found by this technique, for example, mevinolin, lovastatin, and mevacor, which are inhibitors of hydroxymethylglutamyl Coenzyme A reductase, an enzyme involved in cholesterol synthesis. Antibiotics that inhibit glycosyltransferase activities, such as tunicamycin and streptovirudin have also been identified in this manner.

Thus, another important aspect of the present invention is directed to methods for screening samples for ppGalNAcT-1 inhibiting activity. A “sample” as used herein can be any mixture of compounds suitable for testing in a ppGalNAcT-1 assay. A typical sample comprises a mixture of synthetically produced compounds or alternatively a naturally occurring mixture, such as a cell culture broth. Suitable cells include any cultured cells such as mammalian, insect, microbial or plant cells. Microbial cell cultures are composed of any microscopic organism such as bacteria, protozoa, yeast, fungi and the like.

In the typical screening assay, a sample, for example a fungal broth, is added to a standard ppGalNAcT-1 assay. If inhibition or enhancement of activity as compared to control assays is found, the mixture is usually fractionated to identify components of the sample providing the inhibiting or enhancing activity. The sample is fractionated using standard methods such as ion exchange chromatography, affinity chromatography, electrophoresis, ultrafiltration, HPLC and the like. See, e.g., Scopes, Protein Purification, Principles and Practice, 3rd Edition, 1994, Springer-Verlag. Each isolated fraction is then tested for inhibiting or enhancing activity. If desired, the fractions are then further subfractionated and tested. This subfractionation and testing procedure can be repeated as many times as desired.

By combining various standard purification methods, a substantially pure compound suitable for in vivo therapeutic testing can be obtained. A substantially pure modulating agent as defined herein is an activity inhibiting or enhancing compound which migrates largely as a single band under standard electrophoretic conditions or largely as a single peak when monitored on a chromatographic column. More specifically, compositions of substantially pure modulating agents will comprise less than ten percent miscellaneous compounds.

In preferred embodiments, the assays are designed to screen large chemical libraries by automating the assay steps and providing compounds from any convenient source to assays, which are typically run in parallel (e.g., in microtiter formats on microtiter plates in robotic assays).

As noted, the invention provides in vitro assays for ppGalNAcT-1 activity in a high throughput format. For each of the assay formats described, “no inhibitor” control reactions which do not include an inhibitory agent provide a background level of ppGalNAcT-1 activity. In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many different plates per day; assay screens for up to about 6,000-20,000, and even up to about 100,000-1,000,000 different compounds is possible using the integrated systems of the invention. The steps of labeling, addition of reagents, fluid changes, and detection are compatible with full automation, for instance using programmable robotic systems or “integrated systems” commercially available, for example, through BioTX Automation, Conroe, Tex.; Qiagen, Valencia, Calif.; Beckman Coulter, Fullerton, Calif.; and Caliper Life Sciences, Hopkinton, Mass.

In some assays it will be desirable to have positive controls to ensure that the components of the assays are working properly. For example, a known inhibitor of ppGalNAcT-1 activity can be incubated with one sample of the assay, and the resulting increase or decrease in signal determined according to the methods herein.

Essentially any chemical compound can be screened as a potential inhibitor of ppGalNAcT-1 in the assays of the invention. Most preferred are generally compounds that can be dissolved in aqueous or organic (especially DMSO-based) solutions and compounds which fall within Lipinski's “Rule of 5” criteria. The assays are designed to screen large chemical libraries by automating the assay steps and providing compounds from any convenient source to assays, which are typically run in parallel (e.g., in microtiter formats on multiwell plates in robotic assays). It will be appreciated that there are many suppliers of chemical compounds, including Sigma-Aldrich (St. Louis, Mo.); Fluka Chemika-Biochemica Analytika (Buchs Switzerland), as well as numerous providers of small organic molecule libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), Tripos, Inc. (St. Louis, Mo.), Reaction Biology Corp. (Malvern, Pa.), Biomol Intl. (Plymouth Meeting, Pa.), TimTec (Newark, Del.), and AnalytiCon (Potsdam, Germany), among others.

In one preferred embodiment, inhibitors of ppGalNAcT-1 transferase activity are identified by screening a combinatorial library containing a large number of potential therapeutic compounds (potential inhibitor compounds). Such “combinatorial chemical or peptide libraries” can be screened in one or more assays, as described herein, to identify those library members particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.).

Lead compounds that have been identified for their capability to reduce or inhibit the transferase activity of a ppGalNAcT-1 in vitro are then evaluated for their ability to reduce or inhibit a pathological immune and/or inflammatory response in vivo. This can be done using any animal model of a pathological immune responses and/or a pathological inflammatory response known in the art. For example, a pathological inflammatory response can be evaluated using a murine model of acute peritonitis, described herein. In a further example, a pathological immune response can be evaluated using any murine and/or rat models of several autoimmune diseases, including multiple sclerosis (experimental autoimmune encephalomyelitis), rheumatoid arthritis, autoimmune uveitis (experimental autoimmune uveitis), myasthenia gravis (experimental autoimmune myasthenia gravis) known in the art. The ability of a particular compound to prevent, reduce or inhibit manifestations of disease in an animal model can be measured using any known technique. For example, test and control animals can be comparatively tested for disease signs including muscle weakness (e.g., hang test), joint inflammation (e.g., mercury displacement test), and histopathological evidence of tissue destruction (neurological tissue, muscular tissue, ocular tissue, joint tissue, etc.).

4. Therapeutic Uses of the Invention

The compositions and methods of the present invention can be used therapeutically to selectively reduce, inhibit or prevent ppGalNAcT-1 activity that is associated with a variety of pathogenic acute or chronic immune and/or inflammatory responses requiring selectin-based mechanisms (e.g., selectin-mediated leukocyte homing). The conditions can involve cells of the adaptive (e.g., B-cells and/or T-cells) and/or innate (e.g., myeloid cells, including neutrophils) immune systems.

A. Pathogenic Inflammatory Responses

In some embodiments, the methods reduce, inhibit or prevent an undesirable or pathogenic, acute or chronic inflammatory response. The methods find use in treating pathogenic inflammatory responses requiring selectin-mediated mechanisms. The inflammatory responses to be treated can involve cells of the adaptive (e.g., B-cells and/or T-cells) and/or innate (e.g., myeloid cells, including neutrophils) immune systems. Typically, an inflammatory response is initiated by endothelial cells producing molecules that attract and detain inflammatory cells (e.g., myeloid cells such as neutrophils, eosinophils, and basophils) at the site of injury. The inflammatory cells then are transported through the endothelial barrier into the surrounding tissue. The resulting accumulation of inflammatory cells, in particular neutrophils, is followed by generation of toxic oxygen particles and, release of neutrophil granules which contain acid hydrolases and degradative enzymes such as proteases, elastase, and collagenase, which contribute to local tissue breakdown and inflammation. Neutrophils can also release chemoattractants and complement activators that amplify the inflammation.

Although the inflammatory response can play a role in the healing process by destroying, diluting, and isolating injurious agents and stimulating repair of the affected tissue, inflammatory responses can also be harmful, and indeed life-threatening. Five symptoms often characterize the inflammatory response: pain, redness, heat, swelling, and loss of function. For example, inflammation results in leakage of plasma from the blood vessels. Although this leakage can have beneficial effects, it causes pain and when uncontrolled can lead to loss of function and death (such as adult respiratory distress syndrome). Anaphylactic shock, arthritis, and gout are among the conditions that are characterized by uncontrolled or inappropriate inflammation.

Inflammatory responses differ from immune responses mediated by T- and B-lymphocytes in that an inflammatory response is non-specific. While antibodies and MHC-mediated immune responses are specific to a particular pathogen or other agent, the inflammatory response does not involve identification of a specific agent. Both inflammatory responses and specific immune responses, however, involve extravasation of the respective cell types from the blood vessels to the site of tissue injury or infection. Moreover, several of the receptors that mediate extravasation of lymphocytes are also involved in extravasation of inflammatory cells. In particular, lymphocyte trafficking to lymph nodes under normal circumstances is mediated by selectins that are expressed by cells of the vascular endothelium in response to cytokine induction. Selectins are also involved in the recruitment of neutrophils to the vascular endothelium during inflammation (reviewed in Kansas (1996) Blood 88: 3259-87; and McEver and Cummings (1997) J. Clin. Invest. 100: 485-91). Three types of selectins are involved in the interaction between leukocytes and the vascular endothelium. E-selectin (also called endothelial-leukocyte adhesion molecule-1, ELAM-1) and P-selectin are expressed on activated endothelium. P-selectin is also present on activated platelets, while L-selectin is found on lymphocytes. Selectin deficiencies result in varying degrees of impaired lymphocyte trafficking, reduced neutrophil recruitment to sites of inflammation and decreased leukocyte turnover (Arbones et al. (1994) Immunity 1: 247-260; Johnson et al. (1995) Blood 86: 1106-14; Labow et al. (1995) Immunity 1: 709-720; Mayadas et al. (1993) Cell 74: 541-554).

Binding of leukocytes to selectins is at least partially mediated by oligosaccharide ligands that are displayed on the surface of the leukocytes. The oligosaccharide selectin ligands are generally attached to glycoproteins and glycolipids and include Core 1 and Core 2 type O-glycans, for example, sialyl 6-sulfo Lewis x and sialyl Lewis x. Whereas the nonsulfated carbohydrate ligands of selecting, including sialyl Lewis x, are mainly involved in recruitment of leukocytes during inflammation, sulfated carbohydrate ligands of selecting, including sialyl 6-sulfo Lewis x, are mainly involved in routine homing of lymphocytes (see, Kannagi, Curr Opin Struct Biol (2002) 12:599-608).

Leukocyte binding to selectins is followed by additional steps. For example, the transient binding of leukocyte ligands to selectins results in “rolling” or “tethering” in which the leukocytes roll along the surface of the endothelial cells. The leukocytes then receive signals that activate leukocyte integrins into a high affinity state, which results in the leukocytes becoming more firmly bound to the endothelial wall of the blood vessel. Leukocyte extravasation then occurs, a process in which the leukocytes pass through the endothelial wall and enter the underlying tissue.

B. Pathogenic Immune Responses

In some embodiments, the methods reduce, inhibit or prevent an undesirable or pathogenic, acute or chronic, immune response. The methods find use in treating pathogenic immune responses resulting from defective B-cell activity, for example, those that result in overproduction of immunoglobulins or production of pathogenic immunoglobulins against autologous antigens (e.g., B-cell-mediated autoimmune conditions). In some embodiments, the deleterious immune responses involve lymphoid cells, and often do not involve immune cells other than neutrophils. In some embodiments, the invention can be used to inhibit deleterious immune responses associated with autoimmune disease, tissue or organ graft rejection and allergies. Inappropriate activation of the immune system is a component of a number of immunopathologies, including autoimmunity, allograft rejection and allergic responses. Immune mediated pathologies are reviewed, for example, in Chapters 295-301 of Harrison's Principles of Internal Medicine, Kasper, et al., eds., 16th edition, 2005, McGraw-Hill. Exemplary autoimmune diseases include rheumatoid arthritis, multiple sclerosis, myasthenia gravis, autoimmune uveitis, type I diabetes, and systemic lupus erythematosus. The methods find use in treating autoimmune conditions resultant in part from pathogenic B-cell activity (e.g., the generation of autoantibodies), including for example, rheumatoid arthritis, myasthenia gravis, type I diabetes, Hashimoto's thyroiditis, Rheumatic fever, Wegener's granulomatosis, Scleroderma, Sjogren's syndrome and systemic lupus erythematosus. Allergic responses include allergies to various pollens, dust mites and the like. In addition, foreign infectious diseases can cause immunopathology (e.g., lyme disease, hepatitis, LCMV, post-streptococcal endocarditis, or glomerulonephritis). Food hypersensitivities, including celiac disease and Crohn's disease, as well as other allergic diseases, have been associated with inappropriate immune responses or suspected of having an autoimmune component. Other diseases of immune-mediated injury include Rheumatic Fever, Systemic Sclerosis (Scleroderma), Sjögren's Syndrome, Spondyloartiritides, Vasculitis Syndromes, Behçet's Syndrome, Polychondritis, Sarcoidosis and Amyloidosis. These pathological conditions are reviewed, for example, in Chapters 302-310 of Harrison's Principles of Internal Medicine, supra.

Some embodiments of the invention are directed to methods of inhibiting immune responses that are mediated by B lymphocytes, e.g., humoral immunity. These immune responses, in which antibodies recognize and eliminate antigens, are the principal defense mechanism against extracellular microbes and their secreted toxins. B lymphocyte-mediated immune responses are inhibited by interfering with the biosynthesis of selectin ligands (e.g., Core 1 and/or Core 2 type O-glycans). Reducing or inhibiting the transferase activity of ppGalNAcT-1 reduces or inhibits the primary step in selectin ligand formation.

Other embodiments of the invention provide methods of modulating immune responses that are mediated by T lymphocytes, in particular CD8⁺ cytotoxic T lymphocytes (CTL). Such immune responses provide defense against infections by intracellular microbes such as viruses and some bacteria, which proliferate inside host cells and thus are inaccessible to circulating antibodies. CTL responses are also inhibited by interfering with biosynthesis of selectin ligands.

C. Administration

In therapeutic applications, the ppGalNAcT-1 inhibitors of the invention are administered to an individual already suffering from an inappropriate or undesirable immune or inflammatory disorder, for example, an autoimmune disease, an allergic response, or a transplantation rejection response. Compositions that contain ppGalNAcT-1 inhibitors are administered to a patient in an amount sufficient to suppress the undesirable immune and/or inflammatory disorder and to eliminate or at least partially arrest symptoms and/or complications. An amount adequate to accomplish this is defined as a “therapeutically effective dose.” Amounts effective for this use will depend on, e.g., the inhibitor composition, the manner of administration, the stage and severity of the disease being treated, the weight and general state of health of the patient, and the judgment of the prescribing physician. Inhibitors of ppGalNAcT-1 activity can be administered chronically or acutely to treat a particular immune and/or inflammatory disorder. In certain instances, it will be appropriate to administer an inhibitor of ppGalNAcT-1 activity prophylactically, for instance in subjects with a history of immune and/or inflammatory disorders, including autoimmune diseases, allergies or transplantation rejection responses.

Alternatively, DNA or RNA that inhibits expression of one or more sequences encoding a ppGalNAcT-1 enzyme, such as an antisense nucleic acid, a small-interfering nucleic acid (i.e., siRNA), a micro RNA (miRNA), or a nucleic acid that encodes a peptide that blocks expression or activity of a ppGalNAcT-1 can be introduced into patients to achieve inhibition. U.S. Pat. No. 5,580,859 describes the use of injection of naked nucleic acids into cells to obtain expression of the genes which the nucleic acids encode.

Therapeutically effective amounts of ppGalNAcT-1 inhibitor or enhancer compositions of the present invention generally range for the initial administration (that is for therapeutic or prophylactic administration) from about 1.0 mg to about 10 g of ppGalNAcT-1 inhibitor for a 70 kg patient, usually from about 10 mg to about 5 g, and preferably between about 2 mg and about 1 g. Typically, lower doses are initially administered and incrementally increased until a desired efficacious dose is reached. These doses can be followed by repeated administrations over weeks to months depending upon the patient's response and condition by evaluating symptoms of immune cell activity and/or inflammation.

It must be kept in mind that the compositions of the present invention can be employed in severe and/or acute disease states, that is, life-threatening or potentially life threatening situations. In such cases, in view of the minimization of extraneous substances and the relative nontoxic nature of the inhibitors, it is possible and may be felt desirable by the treating physician to administer substantial excesses of these compositions.

For prophylactic use, administration should be given to subjects at risk or those with a history of rogue immune and/or inflammatory disorders. Therapeutic administration may begin at the first sign of disease or the detection or shortly after diagnosis of the immune and/or inflammatory disorder. This is often followed by repeated administration until at least symptoms are substantially abated and for a period thereafter.

The pharmaceutical compositions for therapeutic or prophylactic treatment are intended for parenteral, topical, oral or local administration. Preferably, the compositions are formulated for oral administration. In certain embodiments, the pharmaceutical compositions are administered parenterally, e.g., intravenously, subcutaneously, intradermally, or intramuscularly. Compositions of the invention are also suitable for oral administration. Thus, the invention provides compositions for parenteral administration which comprise a solution of the ppGalNAcT-1 inhibiting agent dissolved or suspended in an acceptable carrier, preferably an aqueous carrier. A variety of aqueous carriers may be used, e.g., water, buffered water, 0.9% saline, 0.3% glycine or another suitable amino acid, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of ppGalNAcT-1 inhibiting agents of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The ppGalNAcT-1 inhibitors of the invention may also be administered via liposomes, which can be designed to target the conjugates to a particular tissue, for example, immune cells, leukocytes, lymphocytes, myeloid cells or endothelial tissues, as well as increase the half-life of the peptide composition. Liposomes include emulsions, foams, micelles, insoluble monolayers, liquid crystals, phospholipid dispersions, lamellar layers and the like. In these preparations, the peptide, nucleic acid or organic compound to be delivered is incorporated as part of a liposome, alone or in conjunction with a molecule which binds to, e.g., a receptor prevalent among the desired cells, or with other therapeutic compositions. Thus, liposomes filled with a desired peptide, nucleic acid, small molecule or conjugate of the invention can be directed to the site of, for example, immune cells, leukocytes, lymphocytes, myeloid cells or endothelial cells, where the liposomes then deliver the selected ppGalNAcT-1 inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid liability and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028.

The targeting of liposomes using a variety of targeting agents is well known in the art (see, e.g., U.S. Pat. Nos. 4,957,773 and 4,603,044). For targeting to desired cells, a ligand to be incorporated into the liposome can include, e.g., antibodies or fragments thereof specific for cell surface determinants of the target cells. A liposome suspension containing a peptide or conjugate may be administered intravenously, locally, topically, etc. in a dose which varies according to, inter alia, the manner of administration, the conjugate being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more conjugates of the invention, and more preferably at a concentration of 25%-75%.

For aerosol administration, the inhibitors are preferably supplied in a suitable form along with a surfactant and propellant. Typical percentages of ppGalNAcT-1 inhibitors are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

An effective immunosuppressive and/or anti-inflammatory treatment is indicated by a decrease in immune and/or inflammatory responses, as measured according to a clinician or by the patient. Alternatively, methods for detecting levels of specific ppGalNAcT-1 activities can be used. Standard assays for detecting ppGalNAcT-1 transferase activity are described herein. Again, an effective immunosuppressive and/or anti-inflammatory treatment is indicated by a substantial reduction in activity of ppGalNAcT-1. As used herein, a “substantial reduction” in ppGalNAcT-1 activity refers to a reduction of at least about 30% in the test sample compared to an untreated control. Preferably, the reduction is at least about 50%, more preferably at least about 75%, and most preferably ppGalNAcT-1 transferase activity levels is reduced by at least about 90% in a sample from a treated mammal compared to an untreated control. In some embodiments, the ppGalNAcT-1 transferase activity is completely inhibited.

5. Transgenic Animals that Lack ppGalNAcT-1

The invention also provides chimeric and transgenic nonhuman animals which contain cells that lack at least one ppGalNAcT-1 gene that is found in wild-type cells of the animal, and methods for producing such animals. These animals are useful for several purposes, including the study of the mechanisms by which O-glycosylation influences immune and/or inflammatory responses. Such “knockout” animals can also be used for producing glycoproteins and glycolipids that, when produced in a wild-type animal, would carry a glycosyl residues that are not desirable for a particular application.

A “chimeric animal” includes some cells that lack the functional ppGalNAcT-1 gene and other cells that do not have the inactivated gene. A “transgenic animal,” in contrast, is made up of cells that have all incorporated the specific modification which renders the ppGalNAcT-1 gene inactive. While a transgenic animal is capable of transmitting the inactivated ppGalNAcT-1 gene to its progeny, the ability of a chimeric animal to transmit the mutation depends upon whether the inactivated gene is present in the animal's germ cells. The modifications that inactivate the ppGalNAcT-1 gene can include, for example, insertions, deletions, or substitutions of one or more nucleotides. The modifications can interfere with transcription of the gene itself, with translation and/or stability of the resulting mRNA, or can cause the gene to encode an inactive ppGalNAcT-1 polypeptide.

The present methods are useful for producing transgenic and chimeric animals of most vertebrate species. Such species include, but are not limited to, nonhuman mammals, including rodents such as mice and rats, rabbits, ovines such as sheep and goats, porcines such as pigs, and bovines such as cattle and buffalo. Methods of obtaining transgenic animals are described in, for example, Pinkert, C A, Ed., Transgenic Animal Technology: A Laboratory Handbook, 2^{nd} Edition, Academic Press, 2003; and Houdebine, Animal Transgenesis and Cloning, John Wiley & Sons, 2003.

One method of obtaining a transgenic or chimeric animal having an inactivated ppGalNAcT-1 gene in its genome is to contact fertilized oocytes with a vector that includes a ppGalNAcT-1-encoding polynucleotide that is modified to contain an inactivating modification. For some animals, such as mice, fertilization is performed in vivo and fertilized ova are surgically removed. In other animals, particularly bovines, it is preferably to remove ova from live or slaughterhouse animals and fertilize the ova in vitro. See DeBoer et al., WO 91/08216. In vitro fertilization permits the modifications to be introduced into substantially synchronous cells. Fertilized oocytes are then cultured in vitro until a pre-implantation embryo is obtained containing about 16-150 cells. The 16-32 cell stage of an embryo is described as a morula. Pre-implantation embryos containing more than 32 cells are termed blastocysts. These embryos show the development of a blastocoel cavity, typically at the 64 cell stage. If desired, the presence of a desired inactivated ppGalNAcT-1 gene in the embryo cells can be detected by methods known to those of skill in the art. Methods for culturing fertilized oocytes to the pre-implantation stage are described by Gordon et al. (1984) Methods Enzymol. 101: 414; Hogan et al. Manipulation of the Mouse Embryo: A Laboratory Manual, C.S.H.L. N.Y. (1986) (mouse embryo); Hammer et al. (1985) Nature 315: 680 (rabbit and porcine embryos); Gandolfi et al. (1987) J. Reprod. Fert. 81: 23-28; Rexroad et al. (1988) J. Anim. Sci. 66: 947-953 (ovine embryos) and Eyestone et al. (1989) J. Reprod. Fert. 85: 715-720; Camous et al. (1984) J. Reprod. Fert. 72: 779-785; and Heyman et al. (1987) Theriogenology 27: 5968 (bovine embryos). Sometimes pre-implantation embryos are stored frozen for a period pending implantation. Pre-implantation embryos are transferred to an appropriate female resulting in the birth of a transgenic or chimeric animal depending upon the stage of development when the transgene is integrated. Chimeric mammals can be bred to form true germline transgenic animals.

Alternatively, the modified ppGalNAcT-1 gene can be introduced into embryonic stem cells (ES). These cells are obtained from preimplantation embryos cultured in vitro. See, e.g., Hooper, M L, Embryonal Stem Cells: Introducing Planned Changes into the Animal Germline (Modern Genetics, v. 1), Int'l. Pub. Distrib., Inc., 1993; Bradley et al. (1984) Nature 309, 255-258. Transformed ES cells are combined with blastocysts from a non-human animal. The ES cells colonize the embryo and in some embryos form the germ line of the resulting chimeric animal. See, Jaenisch (1988) Science 240: 1468-1474. Alternatively, ES cells or somatic cells that can reconstitute an organism (“somatic repopulating cells”) can be used as a source of nuclei for transplantation into an enucleated fertilized oocyte giving rise to a transgenic mammal. See, e.g., Wilmut et al. (1997) Nature 385: 810-813.

The introduction of the modified ppGalNAcT-1 gene into recipient cells can be accomplished by methods known to those of skill in the art. For example, the modified gene can be targeted to the wild type ppGalNAcT-1 locus by homologous recombination. Alternatively, a recombinase system can be employed to delete all or a portion of a locus of interest. Examples of recombinase systems include, the cre/lox system of bacteriophage P1 (see, e.g., Gu et al. (1994) Science 265: 103-106; Terry et al. (1997) Transgenic Res. 6: 349-356) and the FLP/FRT site specific integration system (see, e.g., Dymecki (1996) Proc. Nat'l. Acad. Sci. USA 93: 6191-6196). In these systems, sites recognized by the particular recombinase are typically introduced into the genome at a position flanking the portion of the gene that is to be deleted. Introduction of the recombinase into the cells then catalyzes recombination which deletes from the genome the polynucleotide sequence that is flanked by the recombination sites. If desired, one can obtain animals in which only certain cell types lack the ppGalNAcT-1 gene of interest. See, e.g., Tsien et al. (1996) Cell 87: 1317-26; Brocard et al. (1996) Proc. Nat'l. Acad. Sci. USA 93: 10887-10890; Wang et al. (1996) Proc. Nat'l. Acad. Sci. USA 93: 3932-6; Meyers et al. (1998) Nat. Genet. 18: 136-41).

### EXAMPLES

The following examples are offered to illustrate, but not to limit the claimed invention.

### Example 1

### Experimental Procedures

ppGalNAcT-1 Gene Mutagenesis

Isolation of mouse ppGalNAcT-1 genomic DNA and construction of a targeting vector bearing Cre/loxP recombinant signals was accomplished using a human cDNA probe by procedures previously described (Priatel, J. J. et al., Glycobiology 7:45-56 (1997)). Mice bearing the loxP-flanked exon 3 were bred with Zp3-Cre transgenic mates, as described (Shafi, R. et al., Proc. Natl. Acad. Sci. USA 97:5735-5739 (2000)). Genotyping was performed using Southern blotting and polymerase chain reaction (PCR) by procedures previously published (Priatel, J. J. et al., Glycobiology 7:45-56 (1997)). The wild type ppGalNAcT-1 allele was detected as a 300 bp fragment using primers P1 (5′-TCATCACAGTGTCTACCATGGCTGGAG) and P2 (5′-TTCCAGGACAGCCAGGGCTACACAGAG), while the targeted Ftkneo allele was detected using P1 and P3 (5′-GATCTGATGACCTGTTGTGGACACCTG), yielding a 550 bp fragment.

ppGalNAcT Enzyme Activity

Tissues were extracted and analyzed as described (Hagen, F. K. et al., Glycoconj. J. 12:901-909 (1995)). Enzyme essays for ppGalNAcT-1 activity were performed at 37° C. for 1 to 2 hours using a peptide acceptor PRFQDSSSKAPPPLPSPSRLPG, in a final volume of 25 μl containing 50 μM UDP-GalNAc (77,000 cpm [¹⁴C]), 5 mM AMP, 10 mM MnCl₂, 40 nM Cacodylate, pH 6.5, 40 mM α-mercaptoethanol, and 0.1% Triton X-100. Products were characterized by anion exchange chromatography and evaluated on reverse phase HPLC.

Subcloning and Expression of ppGalNAcT-1 cDNAs

Isolation of cDNAs from wild type and ppGalNAcT-1 exon 3 deletion mice was achieved by RT-PCR amplification using mouse kidney total RNA reverse transcribed with the First Strand cDNA Synthesis Kit (Clontech). The lumenal region of the ppGalNAcT-1 was amplified using the PCR primers Mlu-mT1 (5′CACACGCGTTGCCTGCTGGTGACGTTCTAGAGCTAGT) and BamHI (5′ATGCGGATCCAGCCCAGTCAATCCTTCCTT) to incorporate an Mlu I cloning site into the stem region of the mouse ppGalNAcT-1 isoform. This Mlu I-Bam HI cDNA fragment was cloned into the Mlu I-Bam HI sites of the mammalian expression vehicle pIMIKF3. Three independent ppGalNAcT-1 cDNA clones bearing wild type and A alleles were isolated and characterized. DNA sequence was acquired for all clones. Expression of the recombinant enzymes was achieved by transient transfection of COS7 cells, using Lipofectamine (Life Technologies). All six constructs were transfected in duplicate. Enzyme assays for ppGalNAcT activity were performed at 37° C., using standard assay conditions in a final volume of 25 μl, containing, 500 μM EA2 peptide (PTTDSTTPAPTTK), 50 μM UDPGalNAc (20,000 cpm [¹⁴C]), 10 mM MnCl₂, 40 mM cacodylate, pH 6.5, 40 mM β-mercaptoethanol, and 0.1% Triton X-100.

### Flow Cytometry

Single cell suspension from the spleen, thymus, lymph node, and bone marrow were prepared and red blood cell were removed by ammonium chloride lysis. Cells were incubated in the presence of antibodies (below) in FACS buffer (2% FCS in PBS) for 20 min at 4° C. For E- or P-selectin binding, cells were treated with 0.5 μg/ml of Fc block (anti-CD32/16, Pharmingen) and then incubated with Gr1 antibody as described with and without addition of 5 mM EDTA for 30 min (Maly, P. et al., Cell 86:643-653 (1996)). Cells were washed and incubated with a goat anti-human FITC conjugated secondary antibody (Sigma). Antibodies used were CD3 (2C11), CD4 (RM4-5), CD8 (53-6.7), CD11a (M17/4), CD11b (M1/70), CD18 (C71/16), CD19 (1D3), CD24 (M1/69), CD40 (HM40-3), CD44 (IM7), CD45R/B220 (RA3-6B2), CD62L (MEL-14), B7.2 (GL1), Gr1 (RB6-8C5), I-Ab (AF6-120.1), and PSGL-1 (2PH1). Data were analyzed on a FACScalibur flow cytometer using CELLQUEST software (Becton Dickinson).

### Histology

Frozen sections of lymph nodes were cut at 5 μm, air dried, fixed in acetone, and incubated with biotinylated anti-CD4, biotinylated anti-CD8, and anti-CD45R/B220. After washing, sections were incubated with streptavidin-FITC and goat anti-rat rhodamine-conjugated secondary antibody. For L-selectin binding, sections were incubated with L-selectin-IgG chimera and MECA-79 antibody, and then incubated with goat anti-human IgG FITC-conjugated secondary antibody and goat anti-rat rhodamine-conjugated secondary antibody. The mean fluorescence was analyzed using MetaMorph system (Universal Imaging Corporation).

### Lymphocyte Trafficking

Homing assays were carried out with 2.5×10⁷ cells isolated and incubated with CMFDA (Molecular Bioprobes) prior to injection into the tail vein as previously described (Maly et al., 1996). Lymphoid organs were harvested after 1 hour or 24 hours after injection, and T- and Blymphocytes positive for CMFDA were measured by flow cytometry.

B Cell Activation and Antibody Production B-lymphocytes were purified from splenocytes using the Dynal MPC magnet system (Dynal Biotech). Equivalent numbers of B-cells of each genotype (1×10⁵) were cultured in complete RPMI 1640 medium containing β-mercaptoethanol (0.1 mM), 10% FCS, and L-glutamine with the indicated concentrations of goat F (ab′)2 anti-mouse IgM antiserum (Jackson) or lipopolysaccharide (Sigma). Proliferative capacity was measured by cellular incorporation of 3H-thymidine (2.5 μCi per well) during the last 16 hr of a 72 hr assay period.

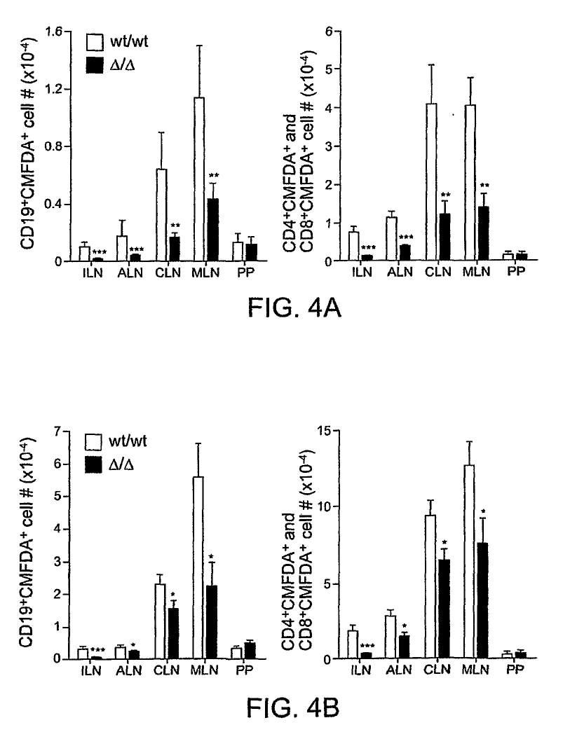

Mice were bled to obtain pre-immune sera and subsequently immunized by intraperitoneal injection of 100 μg of dinitrophenyl (DNP)-keyhole limpet hemocyanin (KLH) (Calbiochem) in Freund's complete adjuvant, or 10 μg of DNP-Ficoll (Biosearch) in PBS. Serum was collected at the indicated times and anti-DNP titers were determined by ELISA using plates coated with 20 μg of DNP-BSA and blocked with 10% FCS in PBS. Mice receiving the DNP-KLH antigen were boosted at the indicated times with the same amount of antigen in Freund's incomplete adjuvant. Sera were diluted to various concentrations and analyzed using anti-mouse isotype-specific antibodies conjugated to alkaline phosphatase (for IgM and IgA, Sigma; for IgG1, IgG2a, IgG2b, and IgG3, Pharmingen). OD405 values were obtained using a microplate reader (Molecular Devices). Results shown in FIG. 4 comprise the indicated sera dilution in the linear range for OD405 values obtained.

### Peritonitis

Mice were administered 1 ml of 0.2% casein in PBS by intraperitoneal injection. At the indicated times, animals were sacrificed and the peritoneal cavities were lavaged with 10 ml of ice-cold PBS containing 1% BSA and 0.5 mM EDTA. Red blood cells were removed by ammonium chloride lysis. Peritoneal cell exudates were incubated with Gr-1 and F4/80 antibodies and analyzed by flow cytometry.

### Statistical Analyses

Data was plotted as the mean ±the standard error of the mean. The number (n) of samples and animals analyzed is provided and the student's t test was used to calculate indicated p values.

### Results

### Germline Mutagenesis of Polypeptide GalNAcT-1 Disrupts O-Glycosylation Activity

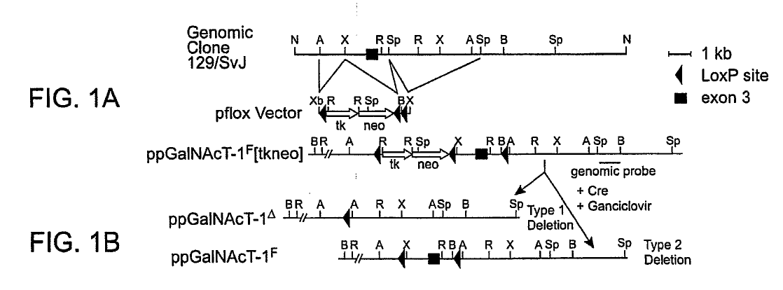

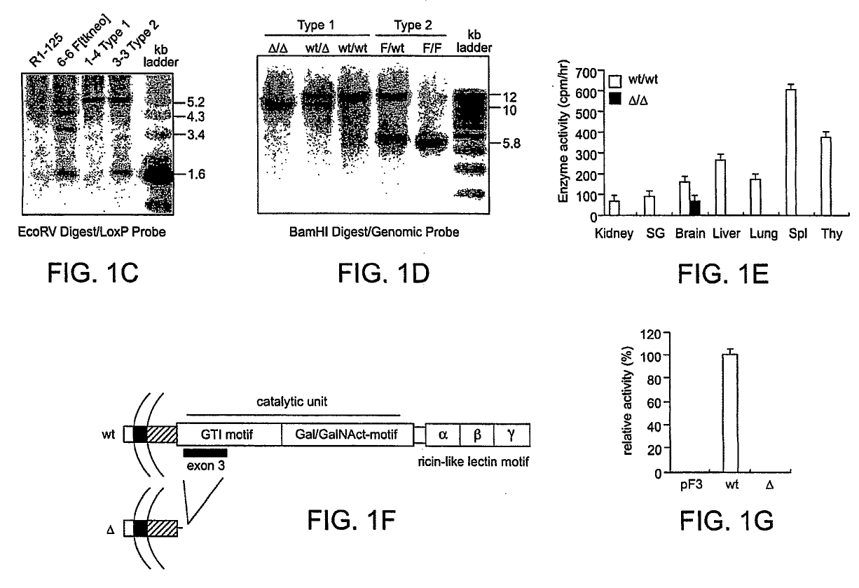

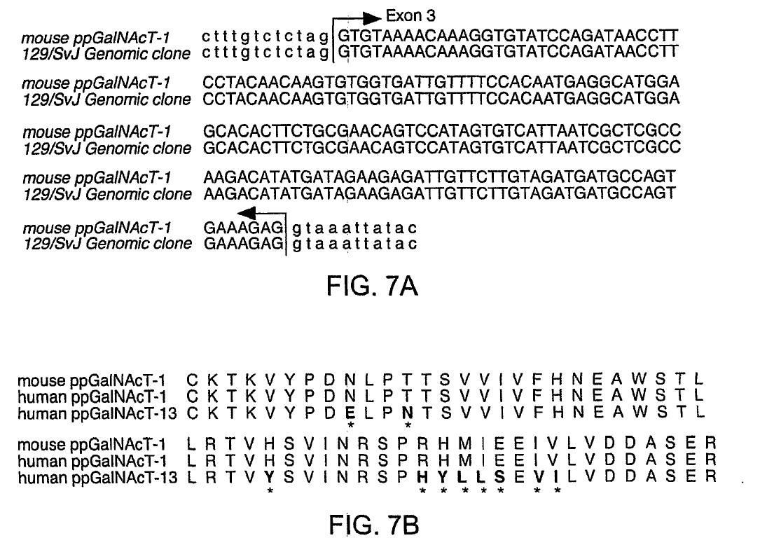

A genomic DNA clone encompassing exon 3 of the mouse gene encoding ppGalNAcT-1 was isolated, characterized by sequence analysis, and used in constructing a gene-targeting vector for conditional mutagenesis (FIG. 1A; FIG. 7). Incorporation of loxP recombination signals flanking exon 3 and the selectable markers permitted modular excision by Cre recombinase activity resulting in the presence of type 1 (A, deleted) and type 2 (F,loxPflanked) alleles (FIG. 1B). Genomic DNA samples from embryonic stem (ES) cells bearing the targeted Ftkneo allele were first characterized for the retention of all three loxP sites (FIG. 1C). Following Cre transfection ES cell sub-clones were isolated bearing the expected type 1 (Δ, deleted) and type 2 (F,loxP-flanked) alleles. Mice bearing the loxP-flanked exon 3 allele (ppGalNAcT-1F) in the germline were generated from ES cell clone 3-3, and were bred with Zp3-Cre transgenic mates as described (Shafi et al., Proc Natl Acad Sci USA. (2000) 97:5735-9) to produce offspring bearing the ppGalNAcT-1Δ allele (FIG. 1D). The ppGalNAcT-1Δ allele was then crossed into the C57BL/6NHsd inbred mouse background for at least 6 generations prior to phenotypic analyses.

A minor but consistent reduction was observed in the frequency of pups homozygous for the ppGalNAcT-1Δ allele; however, those born developed to adulthood without overt physiologic abnormalities and similar to wild-type (wt/wt) and heterozygous (wt/A) littermates. ppGalNAcT-1 enzymatic activity among extracts of multiple tissues of wt/wt and Δ/Δ mice at 8 weeks of age was measured using a peptide substrate that is preferentially O-glycosylated by ppGalNAcT-1. Enzymatic activity was easily detected among multiple tissues from wild-type animals. In contrast, mice homozygous for the ppGalNAcT-1Δ allele were completely deficient of ppGalNAcT-1 activity among all tissues surveyed with the exception of the brain (FIG. 1E). Remaining enzyme activity in brain samples likely reflects expression of the closely related isozyme ppGalNAcT-13 (Hennet, T. et al., Proc. Natl. Acad. Sci. USA 92:12070-12074 (1995); Zhang, Y. et al., J. Biol. Chem. 278:573-584 (2003)). Deletion of exon 3 results in premature translational termination (FIG. 1F). Further confirmation that exon 3 deletion abolishes ppGalNAcT-1 enzyme activity was obtained upon expression of cDNA clones derived from mice bearing wt/wt or Δ/Δ genotypes. Significant enzymatic activity towards peptide substrate in COS7 cell extracts was observed upon high level expression of wild-type ppGalNAcT-1 cDNA while no significant activity could be detected upon similar RNA expression of truncated ppGalNAcT-1 cDNA structure isolated from mice bearing the Δ/Δ genotype (FIG. 1G and data not shown). These findings reveal that deletion of ppGalNAcT-1 exon 3 in the mouse results in an enzymatic null mutation.

Tissue-Specific O-Glycosylation and Selectin Ligand Formation by ppGalNAcT-1 Promotes Lymphocyte Trafficking to Lymph Nodes

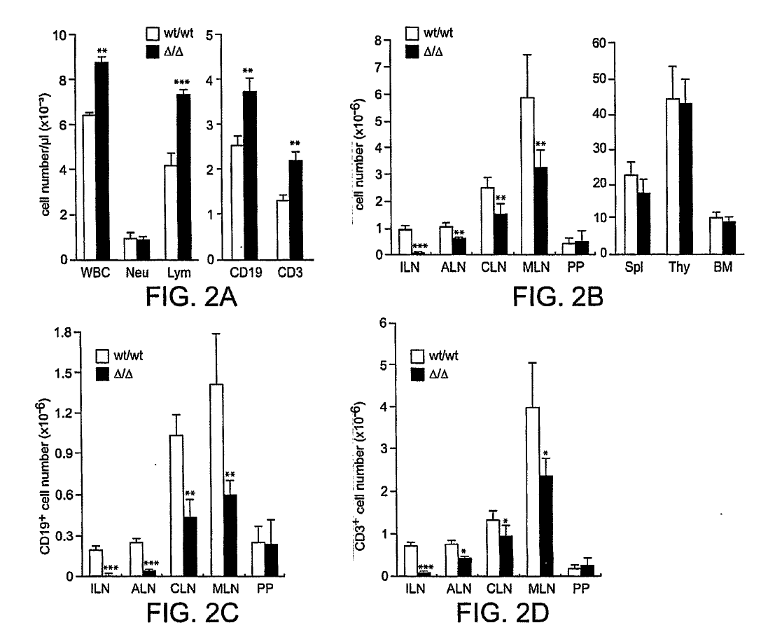

Elevated white blood cell counts were consistently observed among ppGalNacT-1 deficient mice. Both T and B lymphocyte numbers were significantly increased while blood neutrophil levels were unaffected (FIG. 2A). A distinctive profile of altered cellularity was also observed in hematopoietic tissues with a prominent reduction of cell number specifically among lymph nodes, while no alterations were detected in the spleen, thymus, bone marrow, and Peyer's patch (FIG. 2B). Mesenteric, cervical, and axillary lymph nodes were reduced in total cellularity by an average of 60%, while the inguinal lymph node lost 90% of normal cell number and in some cases this lymphoid aggregate was anatomically absent. Characterization of cell populations within the affected lymph nodes revealed deficiencies in both T and B lymphocytes; however, B lymphocytes were most severely reduced in number to 5-50% of normal % (FIG. 2C). T lymphocyte cellularity in comparison among the same lymph nodes was reduced to 30-70% or normal (FIG. 2D).

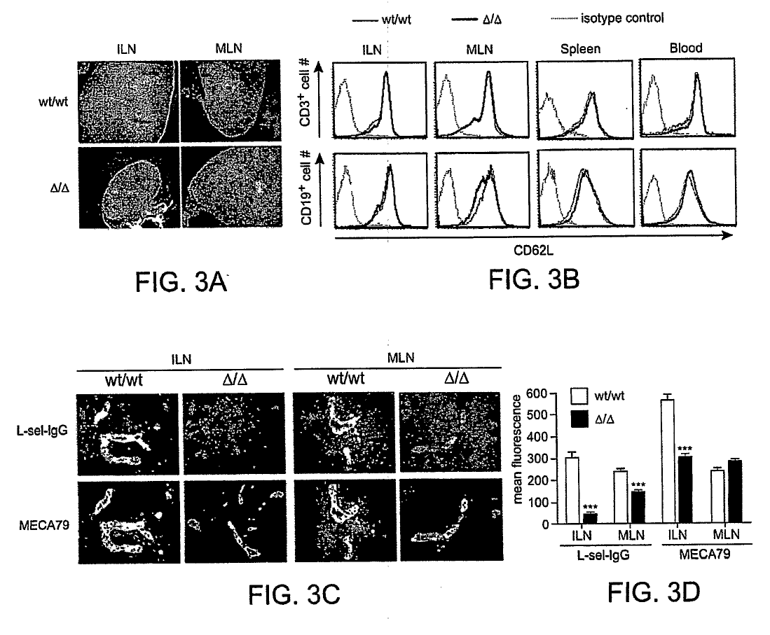

Lymph node architecture comprising cellular and molecular determinants were examined including lymphocyte subpopulations as well as endothelial O-glycans comprising selectin ligands. Significantly reduced anatomic size and numbers of lymph node follicles were invariably found in ppGalNAcT-1 deficiency. Although no change in B and T cell intrafollicular trafficking was suggested, a significant reduction in marginal zone B cell abundance was evident (FIG. 3A). Levels of L-selectin receptors on the surface of T and B lymphocytes were however normal (FIG. 3B). In contrast, L-selectin-IgG chimera binding on high endothelial venules (HEVs) was significantly reduced indicating attenuated expression of L selectin ligands in the absence of ppGalNAcT-1. The inguinal node was the most highly affected with a profound deficiency of HEV L-selectin ligands and a significant decrease in MECA-79 antibody binding (FIG. 3C) indicating that the missing L-selectin ligands are normally O-glycans bearing MECA-79 structures as known to exist on Core 1 O-glycans (Yeh, J. C. et al., Cell 105:957-969 (2001)). Other lymphoid aggregates, including mesenteric lymph nodes, were also deficient in L-selectin ligands but to a lesser extent, while unexpectedly MECA-79 binding levels appeared normal (FIG. 3C). By quantifying fluorescent signals obtained from lymph node HEV, we found that only 10% of L-selectin ligand levels remained among inguinal lymph nodes and 50% of Lselectin ligands remained among mesenteric lymph nodes (FIG. 3D).

Decreased L-selectin ligand formation among lymph node HEV in the absence of ppGalNAcT-1 may explain both the leukocytosis observed and the reduction in colonization of lymph nodes by a decrease in lymphocyte homing. Isolated T and B lymphocytes from wildtype and ppGalNAcT-1 deficient mice were covalently labeled using CMFDA and injected into the tail vein of wild-type recipients. Levels of CMFDA-labeled lymphocytes among lymphoid aggregates were analyzed at 1 hour and 24 hours after transplantation. CMFDA-labeled B and T lymphocytes were detected in all tissues surveyed within 1 hour; however, a significant reduction in homing was observed among wild-type recipients receiving both B and T lymphocytes lacking ppGalNAcT-1 (FIG. 4A). In contrast, at 24 hours post-transplantation, the most severe homing deficits involved the inguinal lymph node, followed by the axillary, cervical, and mesenteric without a reduction in homing to Peyer's Patch tissue (FIG. 4B). Lesser deficits in T cell homing were observed with significant decreased cell number being restricted to inguinal lymphoid aggregates. These findings show that the loss of ppGalNAcT-1 activity reduces lymphocyte homing to lymph nodes with the severity of effect correlated with the extent of HEV selectin ligand deficiency and reduced lymphocyte abundance among the corresponding lymph nodes.

ppGalNAcT-1 Sustains Immunoglobulin-G Production in the Humoral Immune Response

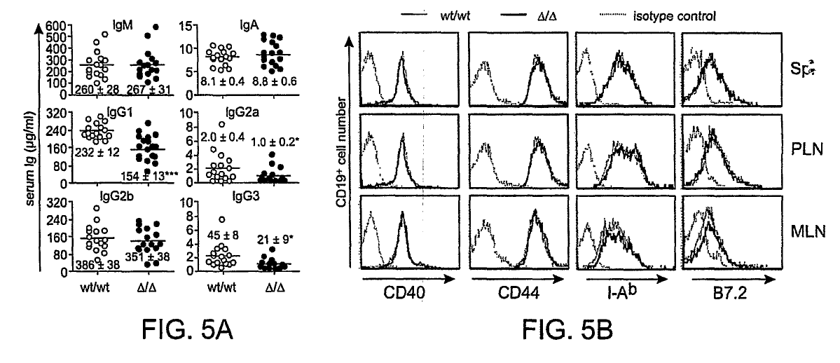

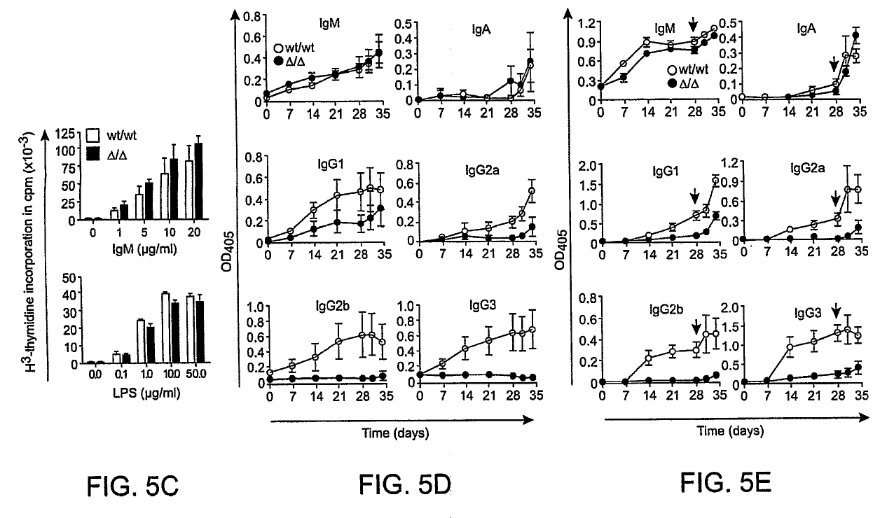

Lymphocyte homing directed by ppGalNAcT-1 contributes to a large proportion of T and B lymphocyte colonization among lymph nodes to the extent that ppGalNAcT-1 may play an important role in acquired immune responses. Circulating levels of immunoglobulins (Ig) in ppGalNAcT-1 deficiency were in fact altered with significant reductions in IgG1, IgG2a, and IgG3 isotypes in the presence of normal IgM and IgA levels (FIG. 5A). B cell surface expression of MHA Class-II and co-stimulatory molecules CD40, CD44, and B7.2 were unaltered in the absence of ppGalNAcT-1. Moreover, B cell proliferation responses to IgM cross-linking or LPS were also unaffected (FIG. 5C). However, immunization with type 2 T-independent antigen DNP-Ficoll resulted in a significant loss of antibody titers representing IgG isotypes in the presence of normal IgM and IgA responses (FIG. 5D). A similar profile with deficit of IgG anti-DNP antibody production was observed following immunizations with the T-dependent antigen DNP-KLH (FIG. 5E). Depressed humoral immunity evident upon immunization in ppGalNAcT-1 deficiency is consistent with requirements for lymph node-dependent cell-cell interactions involving antigen-presentation to B lymphocytes subsequent to the initial immune activation sequence through the IgM antigen receptor complex.

ppGalNAcT-1 Contributes to Neutrophil Selectin Ligand Formation and Inflammation

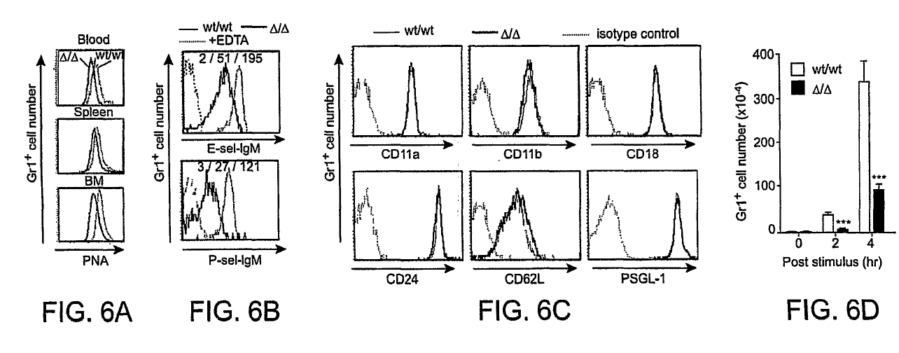

Although circulating myeloid cell numbers were normal in ppGalNAcT-1 deficient mice, this alone does not indicate the presence of normal selectin ligand levels or normal inflammation responses (Ellies, L. G. et al., Blood 100:3618-3625 (2002)). Reduced O-glycosylation at the cell surface was observed among Gr-1+ neutrophils in circulation as well as those in the bone marrow and spleen (FIG. 6A). Neutrophils were further analyzed for the level of E- and P-selectin ligands at the cell surface by flow cytometry. Remarkably, the minor reduction in O-glycan formation was accompanied by a five-fold decrease in E-selectin ligand expression and a 10-fold reduction in P-selectin ligand levels (FIG. 6B). No changes occurred in the expression of other cell surface molecules involved in cell adhesion including CD11, CD18, and L-selectin, as well as selectin counterreceptors CD24 and PSGL-1 (FIG. 6C). The impact of this deficiency among E- and P-selectin ligands on neutrophil recruitment in response to tissue damage and inflammation was explored in a model of acute peritonitis. A significant decrement in intraperitoneal neutrophil recruitment resulted from the absence of ppGalNAcT-1 reflecting a 3-5-fold reduction in cell number (FIG. 6D). This finding is consistent with the observed reduction of E- and especially P-selectin ligand formation due to ppGalNAcT-1 deficiency, indicating that O-glycans contributed by ppGalNAcT-1 further play a significant role in the formation and function of selectin ligands that operate in the innate immune response to activated endothelial cells during inflammation.

### Discussion

A significant proportion of newly synthesized vertebrate proteins transiting the secretory pathway are O-glycosylated by the action of one or more members of a conserved family of ppGalNAcT isozymes. Protein O-glycosylation contributes to mucin structure and function on endothelial cell surfaces, protecting against mechanical stress and pathogen infection (Bansil, R. et al., Annu. Rev. Physiol. 57:635-657 (1995); Tabak, L. A., Annu Rev Physiol. 57:547-64 (1995). These functions of O-glycans rely on the stepwise and hierarchical pattern of peptide modification by multiple ppGalNAcT isozymes in generating a high-density of O-GalNAc linkages that assist in tissue hydration and competition with pathogen receptors. As multiple genes encoding ppGalNAcT isozymes have been selected for and retained during the evolution of multi-cellular organisms it remains possible that key physiologic activities can be attributed to individual ppGalNAcT isozymes upon closer inspection. We have found that ppGalNAcT-1 markedly supports B lymphocyte homing and residency among lymph nodes as well as humoral immunity involving the production of immunoglobulin-G. This role for ppGalNAcT-1 is directly proportional to the level of L-selectin ligands dependent upon this glycosyltransferase among peripheral lymph nodes, and may thereby enhance organism survival during infection and disease. It is possible that other roles for ppGalNAcT-1 exist. In this regard we noticed that a slight decrease in the expected numbers of newborn mice homozygous for the exon 3 deletion were present among litters of heterozygous parents (data not shown).

ppGalNAcT-1 deficiency is likely compensated in large part by other ppGalNAcT isozymes during ontogeny, while the role of ppGalNAcT-1 in adult leukocyte trafficking and humoral immunity is not as effectively compensated.

Selective differences in the homing abilities of T and B lymphocytes have been described independent of their site of origin among spleen, peripheral lymph nodes, mesenteric lymph nodes and Peyer's patches (Stevens, S. K. et al., J. Immunol. 2:844-851 (1982)). L-selectin expression levels appear to be a key contributor to this effect as T lymphocytes express a two-fold higher level of L-selectin than B lymphocyte and are more efficient at homing to peripheral lymph nodes (Stevens, S. K. et al., J. Immunol. 2:844-851 (1982); Tang, M. L. et al., J. Immunol. 160:5113-5121 (1998)). Absence of L-selectin expression on lymphocytes markedly reduces both T and B lymphocyte homing and localization among peripheral lymph nodes as expected, but does not significantly alter lymphocyte numbers in circulation or among mesenteric lymph nodes, spleen, or thymus tissue (Arbones, M. L. et al., Immunity 1:247-260 (1994)). Other distinctions are apparent. Splenomegaly is observed in L-selectin deficiency, but not among ppGalNAcT-1 deficient mice. Remarkably, humoral immune responses were generally increased in the absence of L-selectin. This was attributed to complementary and overlapping roles for the spleen and other lymphoid tissues (Arbones, et al., 1994, supra; and Ley, et al., J Exp Med. 181:669-75 (1995)). Such compensation does not occur in ppGalNAcT-1 deficiency. These findings could imply that L-selectin has additional physiologic roles to play distinct from effects of binding to its glycan ligand. L-selectin ligand synthesis is dependent upon the concerted action of glycosyltransferases and sulfotransferases that together construct 6-sulfo-sialyl Lewis X (sLeX) on glycan branches of several HEV-resident sLeX counter-receptors including GlyCAM-1, CD34, podocalyxin, Sgp200, endoglycan, and MAdCAM-1 (Rosen, S. D. Annu. Rev. Immunol. 22:129-156 (2004)). Glycoproteins bearing sLeX are typically O-glycosylated and have mucin-like domains for multiple O-GalNAc linkages that may act as scaffolds for the production of sLeX. L-selectin ligands can be constructed on Core 1- and Core 2-type O-glycan branches, the former of which also harbors the MECA-79 antigen and is induced in the absence of Core 2 GlcNAcT-1 activity (Yeh, J. C. et al., Cell 105:957-969 (2001)). Three glycosyltransferases have been previously identified as participants in L-selectin ligand synthesis in vivo, specifically Core 2 GlcNAcT-1, FucT-IV, and FucT-VII. Their individual or combined absence has been shown to markedly reduce lymphocyte homing to lymph nodes (Maly, P. et al., Cell 86:643-653 (1996); Ellies, L. G. et al., Immunity 9:881-890 (1998); Homeister, J. W. et al., Immunity 15:115-126 (2001); Gauguet, J. M. et al., Blood 104:4104-4112 (2004)). However, no major reduction in lymph node cellularity has been observed among mice lacking any single glycosyltransferase. In contrast, absence of both FucT-IV and FucT-VII fucosyltransferases results in a profound loss of lymphocyte numbers within peripheral lymph nodes and these animals have impaired T cell-mediated immune responses (Homeister, J. W. et al., Immunity 15:115-126 (2001); Piccio, L. et al., J. Immunol. 174:5805-5813 (2005)). It is not presently known whether these mice harbor a differential loss of B or T lymphocytes among peripheral lymph nodes or exhibit a deficit in humoral immunity. Among Core 2 GlcNAcT-1 deficient mice, a minor (28%) reduction in B cell, but not T cell, numbers among peripheral lymph nodes was observed (Gauguet, J. M. et al., Blood 104:4104-4112 (2004)). However we find that this degree of deficit does not significantly affect immunoglobulin levels in circulation or humoral immunity in assays of antigen-specific antibody production upon immunization (data not shown).

A role in both lymphocyte homing and retention among peripheral lymph nodes has been recently assigned to HEC-GlcNAc6ST (also known as GlcNAc6ST-2 and LSST), a member of the sulfotransferase family (Fukuda, M. et al., J. Biol. Chem. 276:47747-47750 (2001)). Mice lacking this sulfotransferase do not exhibit lymphocytosis; however, a significant deficit of B lymphocytes among peripheral, but not mesenteric, lymph nodes has been detected similar to ppGalNAcT-1 deficiency (Gauguet, J. M. et al., Blood 104:4104-4112 (2004); Hemmerich, S. et al., Immunity 15:237-247 (2001); Hiraoka, N. et al., J. Biol. Chem. 279:3058-3067 (2004)). A role for the HEC-GlcNAc6ST sulfotransferase in humoral immunity has not been reported however at this time. While several genetic loci have been found to affect selectin ligand formation and lymphocyte homing kinetics to lymph nodes, ppGalNAcT-1 is thus far uniquely capable of both supporting B lymphocyte abundance among lymph nodes and promoting humoral immune responses. The role of ppGalNAcT-1 in selectin ligand formation and leukocyte trafficking is focused upon B lymphocyte recruitment and retention among peripheral and mesenteric lymph nodes. Unexpectedly, normal levels of MECA-79 expression were measured among mesenteric lymph nodes of ppGalNAcT-1 deficient mice in the presence of a moderate reduction in L selectin ligands, implying that mesenteric L-selectin ligands generated by ppGalNAcT-1 normally lack the Core 1 O-glycan extension that comprises MECA-79 binding determinants.

The lesser defect in cellularity we observed among mesenteric lymph nodes in ppGalNAcT-1 deficiency may reflect the additional role of MAdCAM-1 as a ligand for the α4β7 integrin expressed on lymphocytes (Berlin et al., 1993). ppGalNAcT-1 contribution to selectin ligand synthesis further supports formation of the majority of counter-receptors for E- and P-selectins on Gr-1+ neutrophils in circulation as judged by expression levels at the cell surface. In comparison to mice lacking Core 2 GlcNAcT-1, there remained slightly higher levels of both E- and P-selectin ligands on neutrophils lacking ppGalNAcT-1 (Ellies, L. G. et al., Immunity 9:881-890 (1998); data not shown). This may explain the lack of neutrophilia in ppGalNAcT-1 deficiency, which exists in the absence of Core 2 GlcNAcT-1, and implies that Core 2 O-glycan-dependent E- and P-selectin ligand synthesis can occur at sites of protein O-glycosylation initiated by other ppGalNAcT isozymes among neutrophils. Nevertheless, loss of ppGalNAcT-1 has a marked impact on neutrophil recruitment in an acute peritonitis model of endothelial inflammation with a degree of reduction in neutrophil influx similar to that seen in animals lacking Core 2 GlcNAcT-1 or FucT-VII.

The initiation of protein O-glycosylation by ppGalNAcT-1 translates the differential expression level of L-selectin on T and B lymphocytes into physiologic alterations primarily supporting B cell retention in secondary lymphoid organs and promoting humoral immunity involving immunoglobulin-G production. No other genetic locus operating in selectin-mediated cell adhesion mechanisms has been identified with similarly vital roles in B lymphocyte retention among peripheral and mesenteric lymph nodes. Both naïve animals and those immunized with the type 2 antigen DNP-Ficoll exhibited significantly reduced IgG isotype titers in the absence of ppGalNAcT-1. Maintenance of lymph node cellularity and IgG isotype production by ppGalNAcT-1 likely reflects the key role of lymph node follicles as sites of effective antigen presentation to B lymphocytes. No change in the kinetics or responses of B lymphocytes upon activation of the B cell antigen receptor was observed in ppGalNAcT-1 deficiency. The in vivo trafficking and homeostatic control of leukocytes are profoundly influenced by O-glycans apportioned by isozyme-specific expression patterns and substrate specificities among multiple members of the ppGalNAcT family, thereby playing a significant role in sustaining humoral immunity.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of inhibiting a pathogenic inflammatory or immune response in a mammal, the method comprising administering to the mammal a compound that inhibits polypeptide GalNAc transferase 1 (ppGalNAcT-1) activity.

2. The method of claim 1, wherein the ppGalNAcT-1 substrate analog is a competitive inhibitor of a ppGalNAcT-1 substrate.

3. The method of claim 3, wherein the compound comprises a ppGalNAcT-1 substrate analog.

4. The method of claim 3, wherein the substrate analog is an analog of a donor substrate.

5. The method of claim 4, wherein the analog of a donor substrate is an analog of UDP-GalNAc.

6. The method of claim 4, wherein the analog of a donor substrate is an analog of UDP.

7. The method of claim 4, wherein the analog of a donor substrate is an analog of GalNAc.

8. The method of claim 3, wherein the substrate analog is an analog of a peptide acceptor substrate.

9. The method of claim 8, wherein the analog of an acceptor substrate is a peptidomimetic.

10. The method of claim 1, wherein the compound is a non-competitive inhibitor.

11. The method of claim 1, wherein the compound is an inhibitory nucleic acid.

12. The method of claim 11, wherein the inhibitory nucleic acid is an antisense RNA molecule.

13. The method of claim 11, wherein the inhibitory nucleic acid is a small interfering RNA (siRNA) molecule.

14. The method of claim 1, wherein the mammal is a human.

15. The method of claim 1, wherein the pathogenic immune response is an autoimmune disorder.

16. The method of claim 1, wherein the autoimmune disorder is selected from the group consisting of rheumatoid arthritis, multiple sclerosis, myasthenia gravis, autoimmune uveitis, and systemic lupus erythematosus.

17. A method of identifying a compound for inhibiting a pathogenic immune or inflammatory disorder in a mammal, the method comprising:
a) providing an assay mixture which comprises: a polypeptide GalNAc transferase 1 (ppGalNAcT-1), a potential immune or inflammatory response inhibitor, a UDP-GalNAc donor saccharide, an acceptor polypeptide, and additional reagents required for ppGalNAcT-1 transferase activity;
b) incubating the assay mixture under conditions in which the ppGalNAcT-1 is active;
c) determining whether the amount of N-acetylgalactosamine transferred to the acceptor polypeptide is decreased in comparison to an assay mixture which lacks the potential immune or inflammatory response inhibitor; and
d) determining whether the potential immune or inflammatory response inhibitor decreases a pathogenic immune or inflammatory disorder in a mammalian disease model for the disorder, whereby a compound for use in inhibiting a pathogenic immune or inflammatory disorder in a mammal is identified.

18. A method of identifying compounds for inhibiting a pathogenic immune or inflammatory disorder in a mammal, the method comprising:
a) providing a cell which comprises a polynucleotide that encodes a ppGalNAcT-1, an acceptor polypeptide for the ppGalNAcT-1, and UDP-GalNAc;
b) contacting the cell with a potential immune or inflammatory response inhibitor and incubating the cell under conditions in which the ppGalNAcT-1 is normally expressed;
c) determining whether the level of a target O-linked glycan moiety is decreased compared to the target O-linked glycan level in the absence of the potential immune or inflammatory response inhibitor; and
d) determining whether the potential immune or inflammatory response inhibitor decreases a pathogenic immune or inflammatory disorder in a mammalian disease model for the disorder, whereby a compound for use in inhibiting a pathogenic immune or inflammatory disorder in a mammal is identified.

19. The method of claim 18, wherein the target O-linked glycan moiety is sialyl 6-sulfo Lewis x or sialyl Lewis x.
